SEQUENCE LISTING (28 sequences)

SEQ ID NO: 1 - protein, 6 aa, Artificial
  Antibody region
  MISC_FEATURE (1)..(6): CDR-H1 6B12
NDYYWN

SEQ ID NO: 2 - protein, 16 aa, Artificial
  Antibody region
  MISC_FEATURE (1)..(16): CDR-H2 6B12
HIGYGGNINYNPSLKN

SEQ ID NO: 3 - protein, 11 aa, Artificial
  Antibody region
  MISC_FEATURE (1)..(11): CDR-H3 6B12
ESFYDGYPFDY

SEQ ID NO: 4 - protein, 11 aa, Artificial
  Antibody region
  MISC_FEATURE (1)..(11): CDR-L1 6B12
RASQDIRNYLN

SEQ ID NO: 5 - protein, 7 aa, Artificial
  Antibody region
  MISC_FEATURE (1)..(7): CDR-L2 6B12
YTSRLHS

SEQ ID NO: 6 - protein, 9 aa, Artificial
  Antibody region
  MISC_FEATURE (1)..(9): CDR-L3 6B12
QQGNSLPRT

SEQ ID NO: 7 - protein, 138 aa, Artificial
  Antibody region
  MISC_FEATURE (1)..(138): VH region 6B12
MKVLSLLYLLTAIPGILSDVQLQESGPGLVKPSQSLSLTCSVTGDSFTNDYYWNWIRQFPGSKLEWMGHIGYGGNINYNPSLKNRISITRDTSKNQFFLRLTSVTTEDTATYYCTRESFYDGYPFDYWGQGTLVTVSA

SEQ ID NO: 8 - DNA, 414 bp, Artificial
  Antibody region
  misc_feature (1)..(414): VH region 6B12
atgaaagtgttgagtctgttgtacctgttgacagccattcctggtatcctgtctgatgtacagcttcaggagtcaggacctggcctcgtgaaaccttctcagtctctgtctctcacctgctctgtcactggcgactccttcaccaatgattattactggaactggatccggcagtttccaggaagcaaactggaatggatgggccacataggctacggcggtaacattaactacaacccatctctcaaaaatcgaatctccatcactcgtgacacatctaagaaccaatttttcctgaggttgacttctgtgactactgaggacacagctacatattactgtacaagagagagtttctatgatggttacccctttgattactggggccaagggactctggtcactgtctctgca

SEQ ID NO: 9 - protein, 127 aa, Artificial
  Antibody region
  MISC_FEATURE (1)..(127): VL region 6B12
MMSSAQFLGLLLLCFQGTRCDIQMTQTTSSLSASLGDRVTISCRASQDIRNYLNWYQQRPGGTLKLLIYYTSRLHSGVPSRFSGSGSGTDYSLTISNLEQEDIATYFCQQGNSLPRTFGGGTKLEIK

SEQ ID NO: 10 - DNA, 381 bp, Artificial
  Antibody region
  misc_feature (1)..(381): VL region 6B12
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattaggaattatttaaactggtatcagcagagaccaggtggaactcttaaactcctgatctactacacatcaagattacactcaggagtcccatcaaggttcagtggcagtgggtctggaacagattattctctcaccattagtaacctggaacaagaagatattgccacttacttttgccaacagggtaattcgcttcctcggacgttcggtggaggcaccaagctggaaatcaaa

SEQ ID NO: 11 - protein, 101 aa, Homo sapiens
MACPLEKALDVMVSTFHKYSGKEGDKFKLNKSELKELLTRELPSFLGKRTDEAAFQKLMSNLDSNRDNEVDFQEYCVFLSCIAMMCNEFFEGFPDKQPRKK

SEQ ID NO: 12 - protein, 24 aa, Homo sapiens
RDNEVDFQEYCVFLSCIAMMCNEF

SEQ ID NO: 13 - protein, 10 aa, Homo sapiens
ELPSFLGKRT

SEQ ID NO: 14 - protein, 11 aa, Homo sapiens
EGFPDKQPRKK

SEQ ID NO: 15 - protein, 5 aa, Artificial
  Antibody region
  MISC_FEATURE (1)..(5): CDR-H1 5C3
ETYMH

SEQ ID NO: 16 - protein, 17 aa, Artificial
  Antibody region
  MISC_FEATURE (1)..(17): CDR-H2 5C3
RIDPANGNTKDDPKFQG

SEQ ID NO: 17 - protein, 6 aa, Artificial
  Antibody region
  MISC_FEATURE (1)..(6): CDR-H3 5C3
SYAMDY

SEQ ID NO: 18 - protein, 16 aa, Artificial
  Antibody region
  MISC_FEATURE (1)..(16): CDR-L1 5C3
RSSQSIVHSNGNTYLE

SEQ ID NO: 19 - protein, 7 aa, Artificial
  Antibody region
  MISC_FEATURE (1)..(7): CDR-L2 5C3
KVSNRLS

SEQ ID NO: 20 - protein, 9 aa, Artificial
  Antibody region
  MISC_FEATURE (1)..(9): CDR-L3 5C3
FQGSHVPFT

SEQ ID NO: 21 - protein, 115 aa, Artificial
  Antibody region
  MISC_FEATURE (1)..(115): VH region 5C3
EAQLQQSGAELVKPGASVKLSCTASGFNIQETYMHWVKQRPEQGLEWIGRIDPANGNTKDDPKFQGKASITVDTSSNTAYLQLSSLTSEDTAVYYCASSYAMDYWGQGTSVTVSS

SEQ ID NO: 22 - protein, 345 aa, Artificial
  Antibody region
  MISC_FEATURE (1)..(345): VH region 5C3
GAGGCTCAGCTGCAGCAGTCTGGGGCAGAGCTTGTGAAGCCAGGGGCCTCTGTCAAGTTGTCCTGCACAGCCTCTGGCTTCAACATTCAAGAGACCTATATGCACTGGGTGAAGCAGAGGCCTGAACAGGGCCTGGAGTGGATTGGAAGGATTGATCCTGCGAATGGTAATACCAAAGATGACCCGAAGTTCCAGGGCAAGGCCTCTATAACAGTAGACACATCCTCCAACACAGCCTACCTGCAGCTCAGCAGCCTGACATCTGAGGACACTGCCGTCTATTACTGTGCTTCAAGTTATGCTATGGACTACTGGGGTCAAGGAACCTCAGTCACCGTCTCCTCA

SEQ ID NO: 23 - protein, 112 aa, Artificial
  Antibody region
  MISC_FEATURE (1)..(112): VL region 5C3
DVLMTQTPLSLPVSLGDQASISCRSSQSIVHSNGNTYLEWYLQKTGQSPELLIYKVSNRLSGVPDRFSGSGSGTDFTLKISRVEAEDLGVYYCFQGSHVPFTFGSGTKLEIK

SEQ ID NO: 24 - protein, 336 aa, Artificial
  Antibody region
  MISC_FEATURE (1)..(336): VL region 5C3
GATGTTTTGATGACCCAAACTCCACTCTCCCTGCCTGTCAGTCTTGGAGATCAAGCCTCCATCTCTTGCAGATCTAGTCAGAGTATTGTACATAGTAATGGAAACACCTATTTAGAATGGTACCTGCAGAAAACAGGCCAGTCTCCAGAGCTCCTGATCTACAAAGTTTCCAACCGACTCTCTGGGGTCCCAGACAGGTTCAGTGGCAGTGGATCAGGGACAGATTTCACACTCAAGATCAGCAGAGTGGAGGCTGAGGATCTGGGAGTTTATTACTGCTTTCAAGGTTCACATGTTCCATTCACGTTCGGCTCGGGGACAAAGTTGGAAATAAAA

SEQ ID NO: 25 - protein, 446 aa, Artificial
  Heavy chain
  misc_feature (1)..(446): AX-202 complete heavy chain
QVQLQESGPGLVKPSQTLSLTCTVSGDSFTNDYYWNWIRQHPGKGLEWIGHIGYGGNINYNPSLKNRLSMSRDTSKNQFSLKLSSVTAADTAVYYCTRESFYDGYPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 214 aa, Artificial
  Light chain
  misc_feature (1)..(214): AX-202 complete light chain
DIQMTQSPSSLSASVGDRVTITCRASQDIRNYLNWYQQKPGKALKLLLYYTSRLHSGVPSRFSGSGSGTDYTLTISSLQPEDFATYYCQQGNSLPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 107 aa, Homo sapiens [sequence omitted]

(19) United States
(12) Patent Application Publication
(10) Pub. No.: US 2023/0220058 A1
(43) Pub. Date: Jul. 13, 2023

(54) Anti-S100A4 antibodies for the treatment of systemic sclerosis

(71) Applicant: Arxx Therapeutics AS, Oslo (NO)

(72) Inventors: Jörg Klingelhöfer, Oslo (NO); Jonas Hallén, Oslo (NO); Rizwan Iqbal Hussain, Oslo (NO); Jörg Hans Wilhelm Distler, Stein (DE); Michal Tomcik, Prague 2 (CZ); Tim Buss, Carlsbad, CA (US); Darragh MacCann, Belfast (GB)

(73) Assignee: Arxx Therapeutics AS, Oslo (NO)

(21) Appl. No.: 18/009,821

(22) PCT Filed: Jun. 30, 2021
(86) PCT No.: PCT/EP2021/068038

§ 371 Date: Dec. 12, 2022

Related U.S. Application Data
(60) Provisional application No. 63/147,485, filed on Feb. 9, 2021.

(30) Foreign Application Priority Data
Jun. 30, 2020 (EP) 20183251.6

(51) Int. Cl. C07K 16/24; A61P 17/00

(52) U.S. Cl. CPC C07K 16/24; A61P 17/00; C07K 2317/76; C07K 2317/31; C07K 2317/24; A61K 2039/505

(57) ABSTRACT

Herein are disclosed anti-S100A4 neutralizing antibodies useful for the treatment of the disease systemic sclerosis. The disclosure also provides isolated polynucleotides, vectors, isolated host cells, compositions and pharmaceutical compositions for the treatment of systemic sclerosis.

### TECHNICAL FIELD

The present invention relates to anti-S100A4 neutralizing antibody molecules and their medical use in the treatment of the disease systemic sclerosis, and more particularly to anti-S100A4 antibody molecules that are capable of inhibiting the biological activity of S100A4, for example in inducing pro-inflammatory signalling and in stimulating TGF-β-induced fibroblast activation and/or collagen synthesis, for the treatment of systemic sclerosis.

### BACKGROUND

Systemic sclerosis (SSc) is a rare connective tissue disease with high morbidity and mortality [1,2]. The hallmarks of the disease are autoimmunity, vasculopathy, sustained inflammation and accumulation of extracellular matrix proteins by pathologically activated fibroblasts [2]. Therapeutic approaches to selectively inhibit the aberrant release of extracellular matrix in SSc are not available to date [1].

The activation of the innate immune system by danger-associated molecular pattern (DAMP) molecules plays a major role in stimulating the pro-inflammatory and pro-fibrotic responses characteristic of SSc [3]. S100A4 is a well-known DAMP which is upregulated upon injury, and a strong inducer of both pro-inflammatory and pro-fibrotic pathways through activation of Pattern Recognition Receptors, including RAGE and TLR-4 [4]. The pro-inflammatory effects are linked to the upregulation of multiple pro-inflammatory cytokines and chemokines such as IL-1β, IL-6, TNF-α, serum amyloid A and CCL5; key inflammatory molecules of innate immunity [5,6,7]. Furthermore, extracellular S100A4 also has a well-documented effect on different elements of the adaptive immune system [8,9,10,11] adding to sustained inflammation, which is important for the pathogenesis of fibrotic disorders [12,13,14].

S100A4 also directly activates pro-fibrotic pathways through interaction with TLR4 which amplifies the transforming growth factor β (TGF-β) driven activation of fibroblasts [22]. TGF-β is a key molecule in the pathogenesis of SSc and IPF and TGF-β signaling is persistently activated in both SSc and IPF where it upregulates the synthesis of collagen in fibroblasts and induces fibrosis in vivo [23]. It has been shown that TGF-β stimulates the expression of S100A4 and in return, S100A4 amplifies TGF-β-induced fibroblast activation and collagen synthesis [22].

Taken together, S100A4 plays an important role in inducing, amplifying and sustaining fibrosis through activation of pro-inflammatory and pro-fibrotic pathways. Directly by the activation of the TGF-β pathway and more indirectly through activation of different immune cells and the secretion of pro-fibrotic cytokines [15]. Numerous clinical studies focused on targeting single molecules either of fibrotic pathways or immune pathways have yielded minimal success.

There is thus a need for a therapeutic approach that can serve the dual role of affecting both the fibrotic and the inflammatory axes that play an important part in the pathophysiology of SSc.

### SUMMARY

The invention is as defined in the claims.

Herein is provided anti-S100A4 neutralizing antibodies for the treatment of systemic sclerosis.

The inventors have tested anti-S100A4 antibodies in two different mouse models for systemic sclerosis and have shown that the antibody treatment is well tolerated and effectively inhibits the pro-fibrotic and pro-inflammatory effects of S100A4 in these disease models. To the best of our knowledge, the invention additionally provides the first therapeutic approach to selectively inhibit accumulation of extracellular matrix proteins by the pathologically activated fibroblasts in systemic sclerosis.

In one aspect, the present invention provides an anti-S100A4 antibody for use in the treatment of systemic sclerosis, wherein the antibody is capable of neutralizing a biological activity of S100A4.

In one aspect, the present invention provides isolated polynucleotides for use in the treatment of systemic sclerosis, which encodes the antibody as described herein.

In one aspect, the present invention provides a vector comprising the isolated polynucleotide as described herein for use in the treatment of systemic sclerosis.

In one aspect, the present invention provides an isolated host cell for use in the treatment of systemic sclerosis comprising the isolated polynucleotide or the vector as described herein.

In one aspect, the present invention provides a pharmaceutical composition for use in the treatment of systemic sclerosis comprising the antibody as described herein together with a pharmaceutically acceptable diluent, carrier and/or excipient.

In one aspect, the present invention provides a composition for use in the treatment of systemic sclerosis comprising the antibody, and the polynucleotide, the vector and/or the cell as disclosed herein

In another aspect, the present invention provides a pharmaceutical composition for use in the treatment of systemic sclerosis, comprising the antibody, the polynucleotide, the vector and/or the cell as disclosed herein, further comprising a pharmaceutically-acceptable diluent, carrier and/or excipient.

### DESCRIPTION OF DRAWINGS

FIG. 1 shows the relative dermal thickness (and subcutaneous adipose layer thickness) of representative images of tissue samples stained with H&E staining from the 5 different sample groups of example 1. Scale bar: 100 μm. The results are further described in Example 1.

FIG. 2 shows the dermal thickness of samples from the 5 different sample groups of example 1. The mean fold change±SEM of 4 determinations per high power field from 8 mice/group is shown in the graph. The value of the NaCl week 1-6 group was set as 1. The Mann-Whitney U-test was used for selected comparisons. The results are further described in Example 1.

FIG. 3 shows the myofibroblast count of samples from the 5 different sample groups of example 1. The mean fold change±SEM is shown in the graph. The value of the NaCl week 1-6 group was set as 1. The Mann-Whitney U-test was used for selected comparisons. The results are further described in Example 1.

FIG. 4 shows the collagen content of the skin of samples from the 5 different sample groups of example 1. The mean fold change±SEM is shown in the graph. The value of the NaCl week 1-6 group was set as 1. The Mann-Whitney U-test was used for selected comparisons. The results are further described in Example 1.

FIG. 5 shows CD-3 positive cell count of samples from the 5 different sample groups of example 1. The mean fold change±SEM is shown in the graph. The value of the NaCl week 1-6 group was set as 1. The Mann-Whitney U-test was used for selected comparisons. The results are further described in Example 1.

FIG. 6 shows the hypodermal thickness of samples from the 4 different sample groups of example 2. The mean fold change±SEM is shown in the graph. P-values are expressed as follows: 0.05>p>0.01 as *; 0.01>p>0.001 as ** as compared to vehicle-treated Tsk1 mice; 0.05>p>0.01 as #; 0.01>p>0.001 as ## as compared to IgG1-treated Tsk1 mice. pa: control mice on the same genetic background not expressing the Tsk1 allele. N=10 for all groups. The results are further described in Example 2.

FIG. 7 shows the myofibroblast counts of samples from the 4 different sample groups of example 2. The mean fold change±SEM is shown in the graph. P-values are expressed as follows: 0.05>p>0.01 as *; 0.01>p>0.001 as ** as compared to vehicle-treated Tsk1 mice; 0.05>p>0.01 as #; 0.01>p>0.001 as ## as compared to IgG1-treated Tsk1 mice. pa: control mice on the same genetic background not expressing the Tsk1 allele. N=10 for all groups. The results are further described in Example 2.

FIG. 8 shows the hydroxyproline content of samples from the 4 different sample groups of example 2. The mean fold change±SEM is shown in the graph. P-values are expressed as follows: 0.05>p>0.01 as *; 0.01>p>0.001 as ** as compared to vehicle-treated Tsk1 mice; 0.05>p>0.01 as #; 0.01>p>0.001 as ## as compared to IgG1-treated Tsk1 mice. pa: control mice on the same genetic background not expressing the Tsk1 allele. N=10 for all groups. The results are further described in Example 2.

FIG. 9 shows the number of CD3-positive T cells of samples from the 4 different sample groups of example 2. The mean fold change±SEM is shown in the graph. P-values are expressed as follows: 0.05>p>0.01 as *; 0.01>p>0.001 as ** as compared to vehicle-treated Tsk1 mice; 0.05>p>0.01 as #; 0.01>p>0.001 as ## as compared to IgG1-treated Tsk1 mice. pa: control mice on the same genetic background not expressing the Tsk1 allele. N=10 for all groups. The results are further described in Example 2.

FIG. 10 shows the effects of monoclonal humanized anti-S100A4 antibodies on fibrotic readouts in bleomycin-challenged mice. Effects of anti-S100A4 antibodies on dermal thickness (A), myofibroblast counts (B) and hydroxyproline content (C). Representative images of HE stained skin sections are shown in D-E. P-values are expressed as follows: 0.05>p>0.01 as *; 0.01>p>0.001 as ** as compared to NaCl; 0.05>p>0.01 as #; 0.01>p>0.001 as ## as compared to mice injected with bleomycin for three weeks followed by injections of NaCl for another 3 weeks. The results are further described in Example 3.

FIG. 11 shows IL-6 secretion by monocytes analysed by Luminex analysis. Data shown is the average of 5 donors. Monocytes purified from PBMC were cultured with media, vehicle, LPS, S100A4 in the absence or presence of mouse IgG1 (A), human IgG4 (B), AX-202 (C) or 6B12 (D) for 6 hours. Data shows levels of IL-6 in the supernatant quantified by Luminex assay. Data presented as mean+SEM arising from five independent donors. “+” indicates at least one donor above the limit of detection for IL-6 (19,200 μg/mL). “−” indicates at least one donor below the limit of detection for IL-6 (8.8 μg/mL). The results are further described in Example 4.

FIG. 12 shows IL-10 secretion by monocytes analysed by Luminex analysis. Data shown is the average of 5 donors. Monocytes purified from PBMC were cultured with media, vehicle, LPS, S100A4 in the absence or presence of mouse IgG1 (A), human IgG4 (B), AX-202 (C) or 6B12 (D) for 6 hours. Data shows IL-10 of cytokine in the supernatant quantified by Luminex assay. Data presented as mean+SEM arising from five independent donors. “−” indicates at least one donor below the limit of detection for IL-10 (8.6 μg/mL). The results are further described in Example 4.

FIG. 13 shows TNF-α secretion by monocytes analysed by Luminex analysis. Data shown is the average of 5 donors. Monocytes purified from PBMC were cultured with media, vehicle, LPS, S100A4 in the absence or presence of mouse IgG1 (A), human IgG4 (B), AX-202 (C) or 6B12 (D) for 6 hours. Data shows levels of TNF-α in the supernatant quantified by Luminex assay. Data presented as mean+SEM arising from five independent donors. “−” indicates at least one donor below the limit of detection for TNF-α (15.20 μg/ml). The results are further described in Example 4.

### DETAILED DESCRIPTION

### Definitions

The term “anti-S100A4 antibody” as used herein encompasses any substantially intact antibody, as well as chimeric antibodies, humanised antibodies, isolated human antibodies, single chain antibodies, polyclonal antibodies, monoclonal antibodies, bispecific antibodies, antibody heavy chains, antibody light chains, homodimers and heterodimers of antibody heavy and/or light chains, and antigen-binding fragments and derivatives of the same, that specifically recognizes an S100A4 antigen. Suitable antigen-binding fragments and derivatives include, but are not necessarily limited to, Fv fragments (e.g. single chain Fv and disulphide-bonded Fv), Fab-like fragments (e.g. Fab fragments, Fab′ fragments and F(ab′)2 fragments), single variable domains (e.g. VH and VL domains) and domain antibodies (dAbs, including single and dual formats [i.e. dAb-linker-dAb]). The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli and cell lines, thus allowing the facile production of large amounts of the said fragments.

The protein S100A4 is also known as 18A2, 42A, CAPL, FSP1, MTS1, P9KA, PEL98 and S100 calcium binding protein A4.

As used herein, the singular forms “a”, “an” and “the” include plural referents unless the context clearly states otherwise. Thus, for example, reference to “an antibody” includes a plurality of such antibodies.

As used herein, the term “variant” defines either a naturally occurring genetic mutant of a DNA sequence or its encoded RNA or protein product, or a recombinantly prepared variation of a DNA sequence or its encoded RNA or protein product. The term “variant” may also refer to either a naturally occurring variation of a given peptide or a recombinantly prepared variation of a given peptide or protein in which one or more amino acid residues have been modified by amino acid substitution, addition, or deletion.

“Inhibition” as used herein means that the presence of the antibody of the invention inhibits, in whole or in part, the binding of ligands to their receptor and/or the disablement of a signal the receptor would elicit upon ligand binding. This includes for example down-stream signalling having effect on cellular behaviour and processes. Also included are other mechanisms of inhibiting the downstream effects of the targeted molecule, such as by blocking dimerization, oligomerization and/or multimerization of the target molecule. “Inhibition”, “blocking” and “neutralizing” are used herein as equivalent terms.

Anti-S100A4 Antibody for the Treatment of Systemic Sclerosis

The present invention relates to anti-S100A4 neutralizing antibodies for use in the treatment of systemic sclerosis.

Thus, it is an aspect of the invention to provide an anti-S100A4 antibody for use in the treatment of systemic sclerosis, wherein the antibody is capable of neutralizing a biological activity of S100A4.

Antibody Target

In some embodiments, the antibody specifically binds to human the S100A4 polypeptide as set forth in SEQ ID NO: 11, preferably to an epitope contained between amino acids 66 and 89 of SEQ ID NO: 11.

In some embodiments, the antibody is capable of binding to an epitope defined by SEQ ID NO: 12. In some embodiments, the antibody is capable of binding to an epitope defined by SEQ ID NO: 13. In some embodiments, the antibody is capable of binding to an epitope defined by SEQ ID NO: 14.

In some embodiments, the antibody is capable of binding to the S100A4 protein in its native conformation.

In some embodiments, the antibody is capable of binding to dimeric, oligomeric and/or multimeric forms of S100A4 protein.

Antibody Composition and Sequence

In some embodiments, the composition of the anti-S100A4 antibody is as described in U.S. Pat. No. 9,683,032.

In some embodiments, the antibody comprises:
a) a heavy chain variable (VH) region comprising:
   i. a CDR-H1 comprising or consisting of the amino acid sequence of SEQ ID NO: 1;
   ii. a CDR-H2 comprising or consisting of the amino acid sequence of SEQ ID NO: 2; and
   iii. a CDR-H3 comprising or consisting of the amino acid sequence of SEQ ID NO: 3;
      and/or
b) a light chain variable (VL) region comprising:
   i. a CDR-L1 comprising or consisting of the amino acid sequence of SEQ ID NO: 4;
   ii. a CDR-L2 comprising or consisting of the amino acid sequence of SEQ ID NO: 5; and
   iii. a CDR-L3 comprising or consisting of the amino acid sequence of SEQ ID NO: 6;
      or a CDR variant of any one of SEQ ID NO:s 1 to 6, wherein any one amino acid has been altered for another amino acid, with the proviso that no more than 3 amino acids have been so altered, for example wherein 2, or 1 amino acids have been so altered in each CDR.

In some embodiments, the heavy chain variable (VH) region of the antibody comprises SEQ ID NO: 7 or a variant thereof having at least 80% identity, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity thereto, and the light chain variable (VL) region of the antibody comprises SEQ ID NO: 9 or variant thereof having at least 80% identity, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity thereto. In some preferred embodiments, the antibody has a VH region defined by SEQ ID NO: 7 and a VL region defined by SEQ ID NO: 9.

In some embodiments, the composition of the anti-S100A4 antibody may be as described in U.S. Pat. No. 9,657,092.

In some embodiments, the antibody comprises:
a) a heavy chain variable (VH) region comprising:
   i. a CDR-H1 comprising or consisting of the amino acid sequence of SEQ ID NO: 15;
   ii. a CDR-H2 comprising or consisting of the amino acid sequence of SEQ ID NO: 16; and
   iii. a CDR-H3 comprising or consisting of the amino acid sequence of SEQ ID NO: 17;
      and/or
b) a light chain variable (VL) region comprising:
   i. a CDR-L1 comprising or consisting of the amino acid sequence of SEQ ID NO: 18;
   ii. a CDR-L2 comprising or consisting of the amino acid sequence of SEQ ID NO: 19; and
   iii. a CDR-L3 comprising or consisting of the amino acid sequence of SEQ ID NO: 20;
      or a CDR variant of any one of SEQ ID NO:s 15 to 20, wherein any one amino acid has been altered for another amino acid, with the proviso that no more than 3 amino acids have been so altered, for example wherein 2, or 1 amino acids have been so altered in each CDR.

In some embodiments, the heavy chain variable (VH) region of the antibody comprises SEQ ID NO: 21 or a variant thereof having at least 80% identity, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity thereto, and the light chain variable (VL) region of the antibody comprises SEQ ID NO: 23 or variant thereof having at least 80% identity, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity thereto. In some preferred embodiments, the antibody has a VH region defined by SEQ ID NO: 21 and a VL region defined by SEQ ID NO: 23.

In some embodiments, the antibody is produced by the ECACC 10022401 hybridoma. In some embodiments, the antibody is produced by the ECACC 11051801 hybridoma. In some embodiments, the antibody is produced by the ECACC 11051802 hybridoma. In some embodiments, the antibody is produced by the ECACC 11051803 hybridoma. In some embodiments, the antibody is produced by the ECACC 11051804 hybridoma. These deposits are described in and are available through U.S. Pat. No. 9,657,092.

The usage of monoclonal antibodies might be useful since they bind to specific binding sites thereby inhibiting access of other molecules to this specific site.

In some embodiments, the antibody is a bispecific antibody.

In some embodiments, the antibody is a complete antibody. In some embodiments, the antibody is a Fab fragment. In some embodiments, the antibody is a F(ab′)₂ fragment. In some embodiments, the antibody is a Fab-like fragment. In some embodiments, the antibody is a scFv. In some embodiments, the antibody is a nanobody, also known as a single-domain antibody. In some embodiments, the antibody is a diabody. In some embodiments, the antibody is a triabody.

In some embodiments, the antibody is an IgG1 subclass antibody. In some embodiments, the antibody is an IgG2 subclass antibody. In some embodiments, the antibody is an IgG3 subclass antibody. In some embodiments, the antibody is an IgG4 subclass antibody. Preferably the subclasses are human IgG subclasses. In some embodiments, the antibody comprises an Fc domain with a mutated IgG constant region.

For the present invention, it may be useful to use an antibody with an immunoglobulin subclass that elicits a weak or no pro-inflammatory response in the host. As shown, the IgG4 subclass may in particular be useful when reduced effector or cross-linking functions of the antibody are desired. In some embodiments, the antibody is therefore a human IgG4 subclass antibody.

In some embodiments, the antibody comprises a human heavy chain constant (CH) region comprising or consisting of the sequence as set forth in SEQ ID NO: 27. In some embodiments, the antibody comprises a CH region comprising or consisting of a variant of SEQ ID NO: 27, said variant having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the antibody comprises a human light chain constant (CL) region comprising or consisting of the sequence as set forth in SEQ ID NO: 28. In some embodiments, the antibody comprises a CL region comprising or consisting of a variant of SEQ ID NO: 28, said variant having at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

In some embodiments, the antibody comprises an Fc domain with a mutated human IgG constant region. In some embodiments, the antibody comprises a mutant human IgG4 heavy chain constant region. In some embodiments, said mutant IgG4 heavy chain constant region comprises an S228P substitution, numbering according to EU numbering. Said S228P substitution may prevent in vivo and in vitro IgG4 Fab-arm exchange, which can result in functionally monovalent, bispecific antibodies (bsAbs) with unknown specificity and therefore, potentially, reduced therapeutic effect. In some embodiments, the terminal lysine of the human IgG4 heavy chain constant region has been removed.

In some embodiments, the antibody is PEGylated.

In some embodiments, the antibody is a humanised antibody. In some embodiments, the antibody is a chimeric antibody. In some embodiments, the antibody is a humaneered antibody.

Antibody Function and Treatment Effects

In some embodiments, the antibody is capable of neutralizing a biological activity of S100A4. Without being bound by theory, S100A4 is thought to stimulate TGF-β-induced fibroblast activation and collagen synthesis, and to mediate pro-inflammatory effects such as T-cell and/or macrophage recruitment and/or infiltration.

In some embodiments, the antibody is capable of inhibiting T-cell and/or macrophage recruitment and/or infiltration mediated by S100A4.

In some embodiments, the antibody is capable of inhibiting the biological activity of S100A4 protein in stimulating cell invasion.

In some embodiments, treatment with the anti-S100A4 antibody reduces fibrosis. Fibrosis may be assessed by measuring dermal thickness, dermal hydroxyproline content, dermal CD3⁺ cell count and/or dermal myofibroblast counts by methods known in the art. Thus, in some embodiments, treatment with the anti-S100A4 antibody reduces dermal thickness, dermal collagen or hydroxyproline content, dermal myoblast count and/or T-cell count.

Antibody Treatment

In some embodiments, the antibody is administered for treatment of systemic sclerosis via parenteral administration such as subcutaneously, intramuscularly or intravenously.

In some embodiments, the antibody is administered every week, such as every 2 weeks, such as every 3 weeks, for example every 4 weeks.

In some embodiments, the antibody is administered once or twice weekly, with a weekly dosage of 5 mg, such as 10 mg, such as 15 mg, such as 20 mg, such as 25 mg, such as 30 mg, such as 40 mg, such as 50 mg, such as 60 mg, such as 75 mg, such as 100 mg, such as 125 mg, such as 150 mg, such as 175 mg, such as 200 mg, such as 225 mg, such as 250 mg, such as 275 mg, such as 300 mg, such as 325 mg, such as 350 mg, such as 375 mg, such as 400 mg, such as 450 mg, such as 500 mg or more.

It may be beneficial to combine treatment with anti-S100A4 antibody together with treatment with other compounds useful for the treatment of systemic sclerosis. Thus, in some embodiments, the antibody is co-administered with another compound for treatment of systemic sclerosis. In some embodiments, the antibody is co-administered with an angiotensin-converting enzyme inhibitor. In some embodiments, the antibody is co-administered with an angiotensin receptor blocker. In some embodiments, the antibody is co-administered with an azathioprine. In some embodiments, the antibody is co-administered with a calcium channel blocker. In some embodiments, the antibody is co-administered with a cyclophosphamide. In some embodiments, the antibody is co-administered with a hydroxychloroquine. In some embodiments, the antibody is co-administered with a mycophenolate. In some embodiments, the antibody is co-administered with a methotrexate. In some embodiments, the antibody is co-administered with a glucocorticoid. In some embodiments, the antibody is co-administered with a phosphodiesterase-5 inhibitor. In some embodiments, the antibody is co-administered with an endothelin receptor antagonist. In some embodiments, the antibody is co-administered with an alpha blocker. In some embodiments, the antibody is co-administered with a prostanoid. In some embodiments, the antibody is co-administered with rituximab. In some embodiments, the antibody is co-administered with a tyrosine kinase inhibitor such as nintedanib. In some embodiments, the antibody is co-administered with tociluzimab.

Polynucleotides, Vectors and Cells Encoding or Expressing the Antibody

In some embodiments, the present invention provides an isolated polynucleotide for use in the treatment of systemic sclerosis, which encodes the antibody as described herein.

In some embodiments, the polynucleotide encoding the heavy chain variable (VH) region of the antibody comprises SEQ ID NO: 8 or a variant thereof having at least 80% identity, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity thereto. In some embodiments, the polynucleotide encoding the light chain variable (VL) region of the antibody comprises SEQ ID NO: 10 or variant thereof having at least 80% identity, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity thereto. In some preferred embodiments, the polynucleotide encoding the antibody encodes a VH region defined by SEQ ID NO: 8 and a VL region defined by SEQ ID NO: 10.

In some embodiments, the polynucleotide encoding the heavy chain variable (VH) region of the antibody comprises SEQ ID NO: 22 or a variant thereof having at least 80% identity, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity thereto. In some embodiments, the polynucleotide encoding the light chain variable (VL) region of the antibody comprises SEQ ID NO: 24 or variant thereof having at least 80% identity, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity thereto. In some preferred embodiments, the polynucleotide encoding the antibody encodes a VH region defined by SEQ ID NO: 22 and a VL region defined by SEQ ID NO: 24.

In some embodiments, the present invention provides a vector comprising the isolated polynucleotide as described herein for use in the treatment of systemic sclerosis.

In some embodiments, the present invention provides an isolated host cell for use in the treatment of systemic sclerosis comprising the isolated polynucleotide or the vector as described herein.

Compositions Comprising the Antibody

In some embodiments, the antibody as described herein may be comprised in a pharmaceutical composition for use in the treatment of systemic sclerosis, together with a pharmaceutically acceptable diluent, carrier and/or excipient. Thus in some embodiments, the present invention provides a pharmaceutical composition for use in the treatment of systemic sclerosis comprising the antibody as described herein together with a pharmaceutically acceptable diluent, carrier and/or excipient.

In some embodiments, the present invention provides a composition for use in the treatment of systemic sclerosis comprising the antibody as disclosed herein, and the polynucleotide, the vector and/or the cell as disclosed herein above in the section “Polynucleotides, vectors and cells encoding or expressing the antibody”.

In some embodiments, the present invention provides a pharmaceutical composition for use in the treatment of systemic sclerosis, comprising the antibody as disclosed herein, and the polynucleotide, the vector and/or the cell as disclosed herein above in the section “Polynucleotides, vectors and cells encoding or expressing the antibody”, and further comprising a pharmaceutically-acceptable diluent, carrier and/or excipient.

### EXAMPLES

### Example 1—Anti-S100A4 Antibody Efficacy in Bleomycin-Induced Dermal Fibrosis

Materials and Methods

Mouse Model of Bleomycin-Induced Skin Fibrosis

The underlying molecular mechanisms in this model are similar to that observed in SSc with infiltration of T cells and macrophages, which activate the resident fibroblasts and induce epithelial-to-mesenchymal transition via the release of pro-fibrotic cytokines. This model is useful to evaluate early inflammatory stages of the disease. In brief, after shaving a 1 cm² of the skin in the upper back area skin fibrosis was induced in 6-week-old C57BL/6J male mice by subcutaneous (s.c.) injection of 0.5 mg/mL bleomycin (BLM) every other day for 6 weeks. Three groups of mice challenged by BLM were intraperitoneally (i.p.) injected each with 100 to 200 μL of following concentration of S100A4-neutralizing antibody (SNA): 2.5, or 7.5 mg/kg/d, started on day 22 and continued every third day for 3 weeks. For the control, 100 μL NaCl was injected s.c. every other day for 6 weeks. For the vehiculum control BLM-challenged mice were i.p. injected with 100 μL PBS, started on day 22 and continued every third day for 3 weeks. A further control was included to determine skin fibrosis upon withdrawal of the BLM-challenge. For this 3-week BLM-challenged mice were injected by both, (i) 100 μL NaCl s.c. started on day 22 every other day for 3 weeks and (ii) vehiculum control PBS i.p. Mice were sacrificed at day 42, and skin, blood and lungs were collected for analysis. Dermal thickness, collagen content as well as numbers of myofibroblasts and CD3⁺ cells in the skin tissue were assessed.

The following groups with each N=8 mice were analyzed:

Group 1: NaCl (w 1-6) s.c.

Group 2: BLM (w 1-6) s.c.+PBS i.p. (w 4-6)

Group 3: BLM (w 1-3) s.c.+NaCl (w 4-6) s.c.+PBS i.p. (w 4-6)

Group 4: BLM (w 1-6) s.c.+SNA (w 4-6) 2.5 mg/kg i.p.

Group 5: BLM (w 1-6) s.c.+SNA (w 4-6) 7.5 mg/kg i.p.

BLM was dissolved in 0.9% NaCl in a concentration of 0.5 mg/mL. The antibodies were diluted in sterile PBS and injected i.p. in a volume of 100 uL for group 4 and 5 and 200 μL for group 6, respectively. The anti-S100A4 antibody which was shipped to the study site had following concentration of 2 mg/mL, batch #Lot 003P10T120116JKL and date January 2016.

Animals were observed regularly (at every animal house visit) for their vital signs, activity, quality and texture of their fur. In addition, mice were weighted weekly on a calibrated scale on the same day and hour from right before the start of BLM administration, till right before their sacrifice. Upon autopsy of mice, all internal organs were screened for macroscopically visible pathologies (e.g. tumors, hemorrhages etc.). Prior to the commencement of experiments, all 48 mice were randomly allocated into 6 cages by the personnel of the animal house, who was blinded to the aims of this project.

Quantification of Dermal Thickening

The injected skin areas were excised, subsequently fixed in 4% formalin for 6 h, thereafter in 70% ethanol, and embedded in paraffin. Skin sections were cut and stained with hematoxylin/eosin. Dermal thickness at the injection sites was analyzed with a BX53 microscope with a DP80 Digital Microscope Camera and CellSens Standard Software (Olympus, Philadelphia, Pa., USA) at 100-fold magnification. The dermal thickness was determined by measuring the largest distance between the epidermal-dermal junction and the dermal-subcutaneous fat junction as described [16]. The dermal thickness was quantified in 4 different sections from different sites with 4 measurements per section. The analysis was performed in a blinded manner.

Quantification of Fibrosis—Masson's Trichrome Staining

The paraffin embedded skin sections were stained with Blue Masson's Trichrome to better visualize the collagen bundles in the dermis, which stain blue. Stained skin sections were visualized with a BX53 microscope with a DP80 Digital Microscope Camera and CellSens Standard Software (Olympus, Philadelphia, Pa., USA) at 100-fold magnification.

Collagen Assessment by Hydroxyproline Assay

The amount of collagen protein in skin samples was determined via hydroxyproline assay. After digestion of full skin thickness punch biopsies (Ø4 mm, two samples/animal) derived from the upper back in 6 M HCl for three hours at 120° C., the pH of the samples was adjusted to 6 with 6 M NaOH. Afterwards, 0.06 M chloramine T was added to each sample and incubated for 20 min at room temperature. Next, 3.15 M perchloric acid and 20% p-dimethylaminobenzaldehyde were added and samples were incubated for additional 20 min at 60° C. The absorbance was determined at 557 nm with a Spectra MAX 190 microplate spectrophotometer (Molecular Devices) for each of the two full skin thickness punch biopsies.

Detection of Myofibroblasts

Myofibroblasts are characterized by the expression of α-smooth muscle actin (αSMA). Fibroblasts positive for αSMA were detected in paraffin-embedded skin sections from the upper back by incubation with monoclonal mouse anti-αSMA antibodies (Merck, A5228). The right concentration of the antibody for the staining of the antibody was determined by dilution row experiment and reviled a dilution of 1:1000 with a concentration of 2 μg/mL. The expression was visualized with horseradish peroxidase labeled secondary antibodies (polyclonal goat anti-mouse IgG/HRP, Dako, P0447, 1:200, with a concentration of 5 μg/mL) and 3,3-diaminobenzidine tetrahydrochloride (DAB) (Sigma-Aldrich). Negative Control Mouse-Coctail of mouse IgG1, IgG2a, IgG2b, IgG3 and IgM Ready to use (Dako, IS750) was used for controls. The sections were then counterstained with Mayer's hematoxylin solution and visualized with a BX53 microscope with a DP80 Digital Microscope Camera and CellSens Standard Software (Olympus, Philadelphia, Pa., USA) at 100- and 200-fold magnification. The analysis was performed by a blinded reviewer counting the number of the myofibroblasts in the full thickness of the dermis (i.e. spindle shaped fibroblasts with hematoxylin-stained nucleus and strong positive staining for αSMA in the cytoplasm) in four sections per sample.

Detection of T Cells

CD3 is a multimeric protein complex serving as a T cell co-receptor. Being a defining feature of the T cell lineage, CD3 can be used as a marker for T cells. Cells positive for CD3 were detected in paraffin-embedded skin sections from the upper back by incubation with polyclonal rabbit anti-CD3 antibodies (Abcam, Ab5690). The right concentration of the antibody for the staining of the antibody was determined by dilution row experiment and reviled a dilution of 1:50 with a concentration of 4 μg/mL. The expression was visualized with horseradish peroxidase labeled secondary antibodies (Polyclonal goat anti-rabbit IgG/HRP, Dako, P0448, 1:200, with a concentration of 1.25 μg/mL) and 3,3-diaminobenzidine tetrahydrochloride (DAB) (Sigma-Aldrich). Negative Control Rabbit Ig fraction of serum from non-immunized rabbits, solid phase absorbed Ready to use (Dako, IS600) were used for controls. The sections were then counterstained with Mayer's hematoxylin solution and visualized with a BX53 microscope with a DP80 Digital Microscope Camera and CellSens Standard Software (Olympus, Philadelphia, Pa., USA) at 100- and 200-fold magnification. The analysis was performed by a blinded reviewer counting the number of the mononucleated (with hematoxylin-stained nucleus) circle-shaped cells stained positive for CD3 in the perivascular infiltrates of the subcutaneous tissue in four sections per sample.

Statistics

All data are presented as mean±SEM. Normal distribution was of data was tested by Kolmogorov-Smirnov test and thereafter, differences between the groups for non-related samples were tested for their statistical significance by parametric unpaired t test or non-parametric Mann-Whitney U test. Statistical analysis and graphs were created using GraphPad Prism 5 (version 5.02; GraphPad Software, La Jolla, Calif., USA). P-values less than 0.05 were considered significant. P-values are expressed as follows: 0.05>p>0.01 as *; 0.01>p>0.001 as **; p<0.001 as ***.

Ethical Statement

Animal studies were conducted in accordance with Act No. 246/1992 Coll. (Czech Republic) Decree No. 419/2012 Coll. (Czech Republic) guidelines for the care and use of laboratory animals and approved by the Ethics Committee of the Institute of Rheumatology in Prague, ref. number of the approved project 5689/2015.

Results

Both concentrations of anti-S100A4 antibody were well tolerated without obvious signs of toxicity on clinical examination or on gross necropsy.

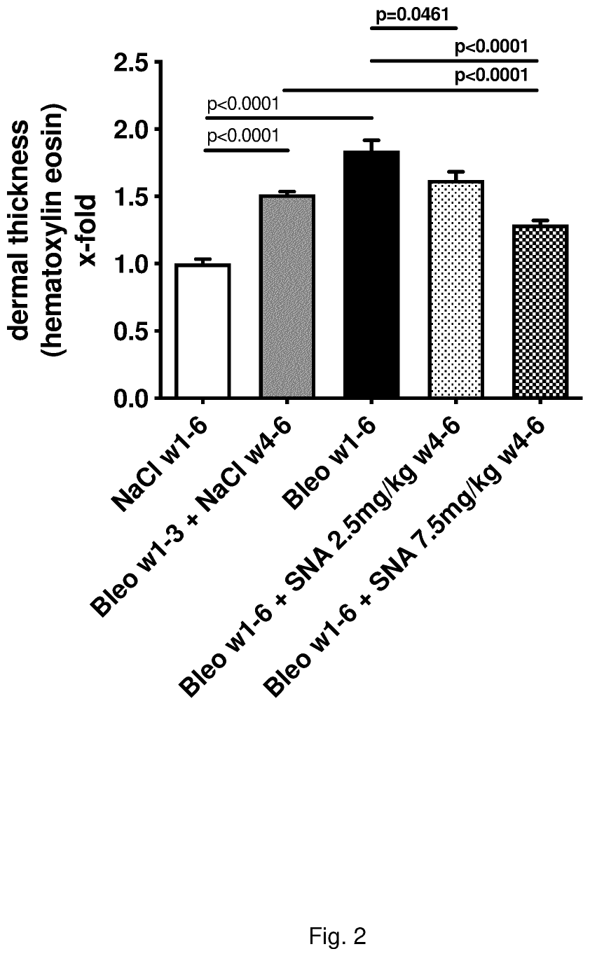

BLM induced prominent skin fibrosis in the exposed skin area with dermal thickening, myofibroblast differentiation and proliferation and increased hydroxyproline content that progressed during treatment. The withdrawal of BLM in the last three weeks of the experiment (with PBS control) showed reduced fibrotic parameters: dermal thickening, myofibroblast differentiation and hydroxyproline content was reduced by 18%, 33% and 11%, respectively when compared to the group challenged with BLM for 6 weeks (as seen in FIGS. 2 to 4).

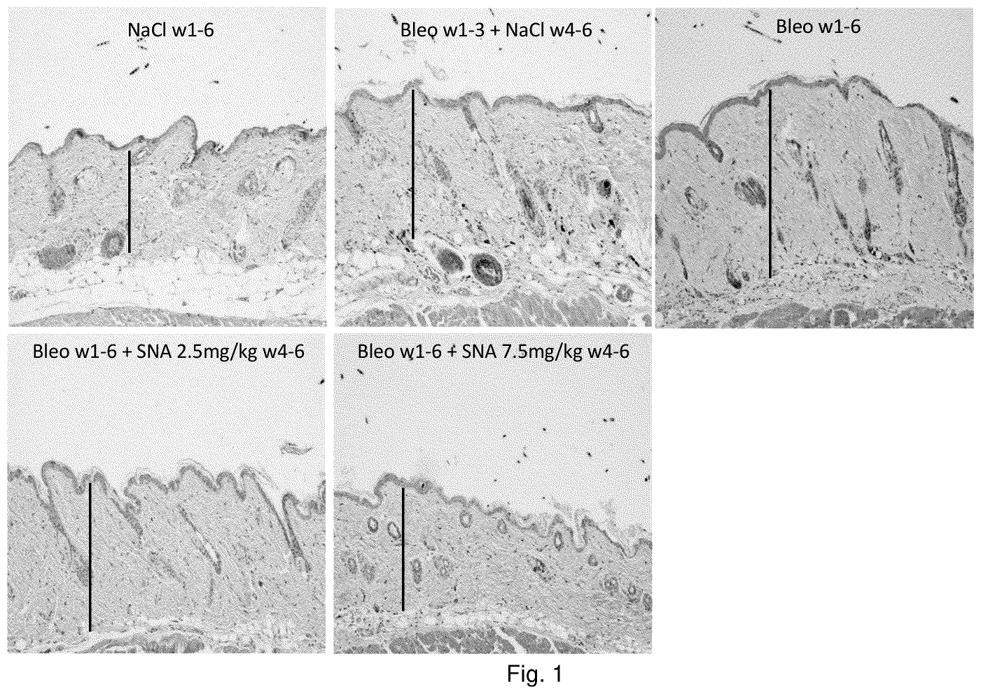

Only the 7.5 mg/kg concentration of anti-S100A4 antibody significantly reduced dermal thickening, myofibroblast counts, hydroxyproline content and number of cells positive for CD3 as compared to vehicle-treated group of mice challenged with bleomycin for 6 weeks (as seen in FIGS. 1 to 5).

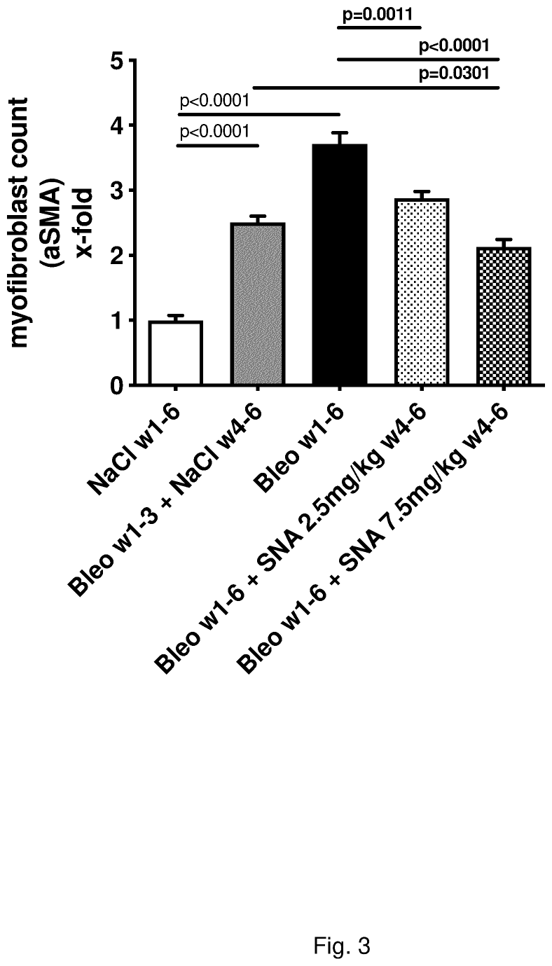

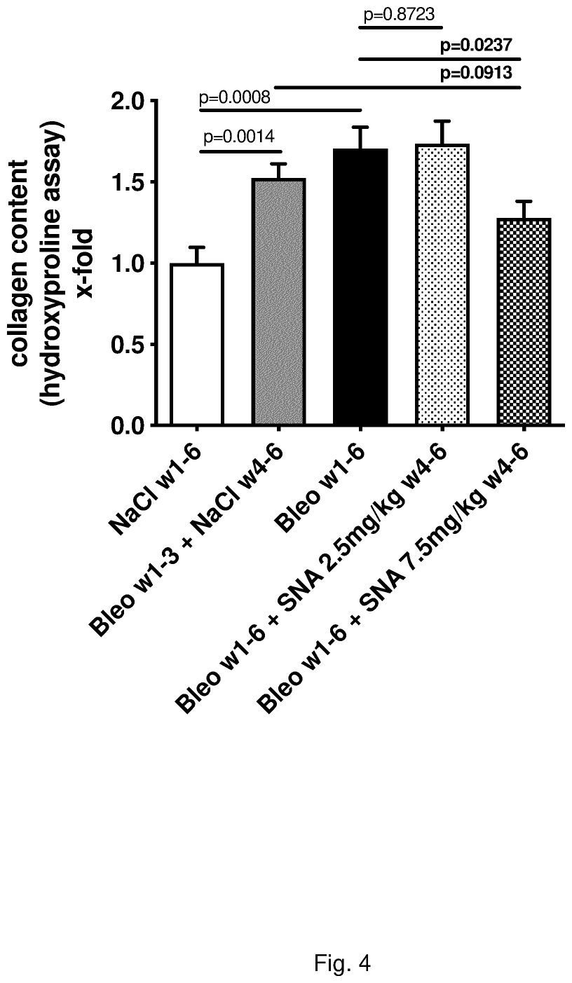

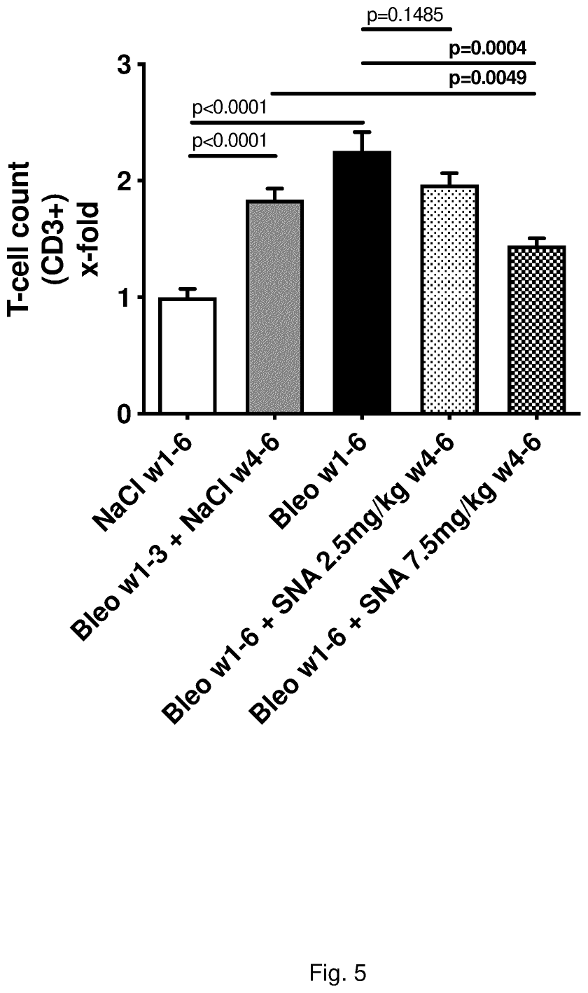

Treatment with 7.5 mg/kg anti-S100A4 antibody reduced the dermal thickness (as seen in FIGS. 1 and 2), number of myofibroblasts (as seen in FIG. 3), hydroxyproline content (as seen in FIG. 4) as well as the number of CD3 positive cells to levels below BLM-withdrawal, i.e. mice challenged with BLM for the first three weeks and then injected with NaCl, (as seen in FIG. 5), thus suggesting a reduction to the pre-treatment levels of fibrosis. These observed effects in this group thus suggest therapeutic efficacy of 7.5 mg/kg anti-S100A4 antibody in both prevention of progression of dermal fibrosis and treatment of pre-established dermal fibrosis induced by repetitive subcutaneous injections of bleomycin in mice.

Only a modest effect in the group of mice treated with the lowest concentration of anti-S100A4 antibody, i.e. 2.5 mg/kg was demonstrated in reducing dermal thickness and myofibroblast count below the levels of mice challenged with BL for 6 weeks and treated with vehicle (as seen in FIGS. 2 and 3).

In conclusion, the results indicate that inhibition of S100A4 ameliorates skin fibrosis in BLM-induced scleroderma model.

### Example 2—Targeting of S100A4 in the Tight-Skin-1 Model of Systemic Sclerosis

Materials and Methods

Tight-Skin-1 (Tsk-1) Model

Tsk-1 mice carry a heterozygous mutation in the fibrillin-1 gene. Littermates carrying two wild type alleles were used as controls. Treatment of Tsk-1 mice was started at an age of 5 weeks and continued until week 10. Mice were sacrificed at an age of 10 weeks and the outcome was analyzed as described below. The following groups with n=10 mice each (5 males and 5 females in ach group) were analyzed:
Non-fibrotic pa mice not carrying the Tsk-1 allele treated with vehicle (PBS)
Tsk-1 mice treated with vehicle
Tsk-1 mice treated with anti-S100A4 antibodies (7.5 mg/kg, 2 mg/ml; injection every Monday, Wednesday and Friday)
Tsk-1 mice treated with isotype mIgG1 (2 mg/ml; Lot nr: 722919A2; injection every Monday, Wednesday and Friday)

Quantification of Hypodermal Thickening

Defined areas of the skin of the upper back were excised, subsequently fixed in 4% formalin for 6 h and embedded in paraffin. Skin sections were cut and stained with hematoxylin/eosin. The hypodermal thickness (measured as distance between the muscle layers surrounding the hypodermis in arbitrary units) was quantified in four different sections from different sites with two measurements per section as described [2,17-21]. The analysis was performed in a blinded manner.

Detection of Myofibroblasts

Myofibroblasts are characterized by the expression of α-smooth muscle actin (αSMA). Fibroblasts positive for αSMA were detected in paraffin-embedded slides from the upper back by incubation with monoclonal anti-αSMA antibodies (clone 1A4, Sigma-Aldrich, Steinheim, Germany). The expression was visualized with horseradish peroxidase labeled secondary antibodies and 3,3-diaminobenzidine tetrahydrochloride (DAB) (Sigma-Aldrich). Monoclonal mouse IgG antibodies (Calbiochem, San Diego, Calif., USA) were used for controls. The analysis was performed by a blinded reviewer (JD) evaluating the myofibroblasts in four sections per sample.

Hydroxyproline Assay

The amount of collagen protein in skin samples was determined via hydroxyproline assay. After digestion of full skin thickness punch biopsies (Ø3 mm) derived from the upper back in 6 M HCl for three hours at 120° C., the pH of the samples was adjusted to 6 with 6 M NaOH. Afterwards, 0.06 M chloramine T was added to each sample and incubated for 20 min at room temperature. Next, 3.15 M perchloric acid and 20% p-dimethylaminobenzaldehyde were added and samples were incubated for additional 20 min at 60° C. The absorbance was determined at 557 nm with a Spectra MAX 190 microplate spectrophotometer.

Staining for CD3/T Cells

Formalin-fixed, paraffin-embedded skin sections were stained with rat anti-CD3 antibodies (ab11089, Abcam, Cambridge, UK). Alexa Fluor-conjugated donkey anti-rat antibodies (Life Technologies, Darmstadt, Germany, A21208). Isotype rabbit control antibodies served as controls. Sections were counterstained with DAPI to visualize nuclei. Positive cells were counted by an experienced reviewer.

Statistics

All data are presented as mean±SEM, and differences between the groups were tested for their statistical significance by Mann-Whitney U non-parametric test for non-related samples. P-values less than 0.05 were considered significant. P-values are expressed as follows: 0.05>p>0.01 as *; 0.01>p>0.001 as **; p<0.001 as *** as compared to vehicle-treated, 10-week-old Tsk1 mice.

Results

Treatment with anti-S100A4 antibodies was well tolerated without obvious signs of toxicity on clinical examination, on gross necropsy or on histology.

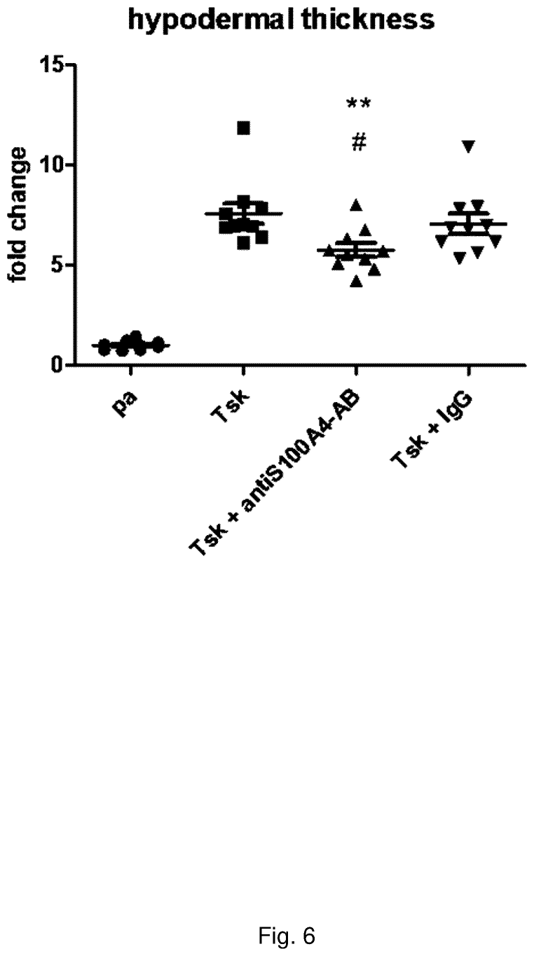

Tsk-1 mice developed prominent skin fibrosis with hypodermal thickening, myofibroblast differentiation and increased hydroxyproline content (as seen in FIGS. 6 to 9).

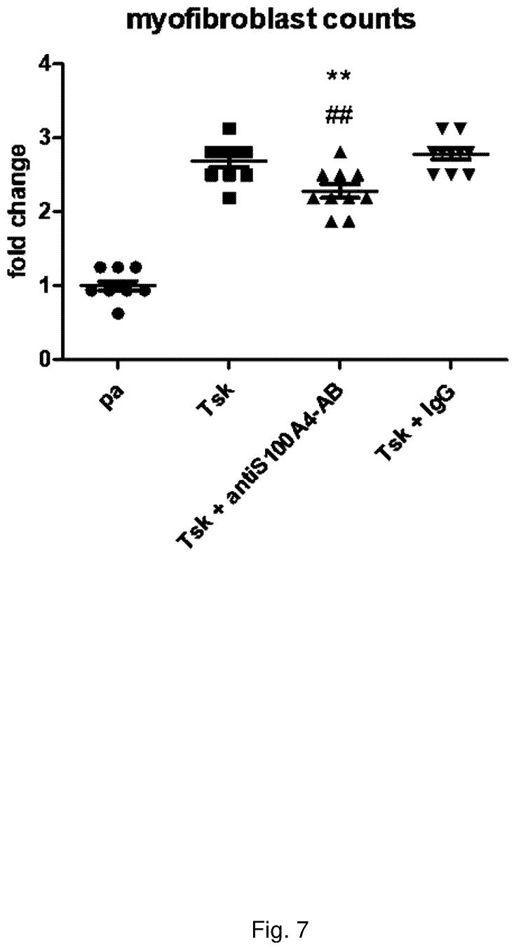

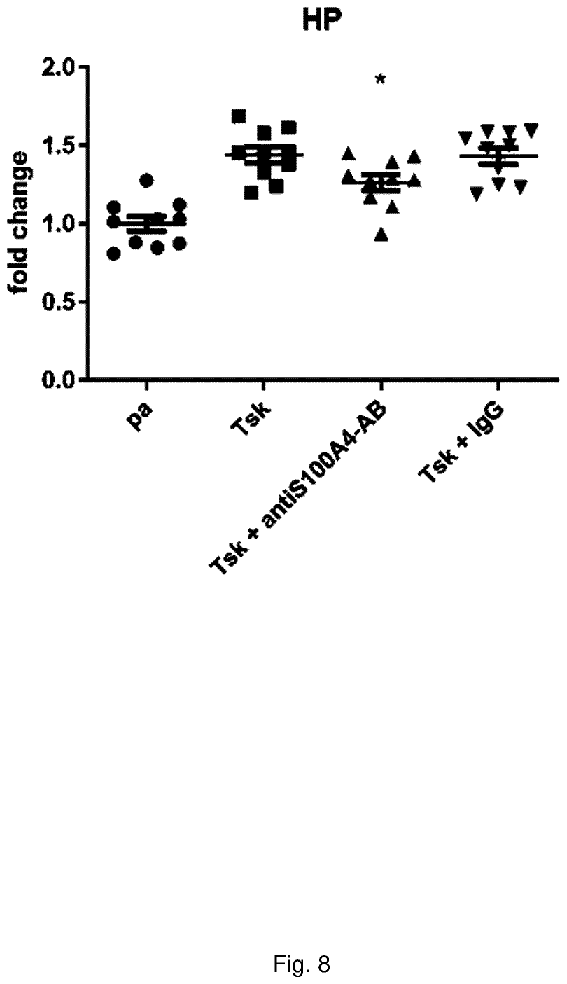

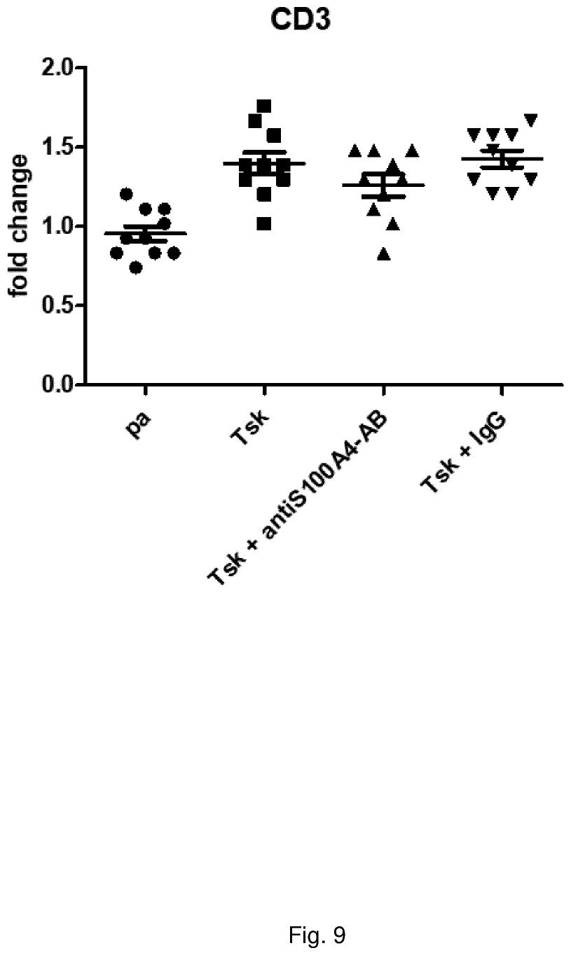

Treatment with anti-S100A4 antibodies significantly reduced hypodermal thickening (as seen in FIG. 6), myofibroblast counts (as seen in FIG. 7) and the hydroxyproline content (as seen in FIG. 8) as compared to vehicle-treated, 10-week-old Tsk1 mice or compared to Tsk-1 mice treated with IgG1. A trend towards decreased T cell counts was also observed in anti-S100A4-treated mice (as seen in FIG. 9), which, however, failed to reach statistical significance. This might be due to the rather small differences in T cell counts between Tsk mice and control mice.

In conclusion, the results indicate that treatment with anti-S100A4 antibodies ameliorates skin fibrosis in Tsk-1 mice.

### Example 3—Humanized Anti-S100A4 Antibody Efficacy in Treating Bleomycin-Induced Dermal Fibrosis In Vivo

Systemic sclerosis (SSc) is a systemic fibrosing orphan disease with high morbidity and mortality. SSc is the condition with the highest case specific mortality of any of the autoimmune rheumatic diseases with more than half of cases diagnosed with the condition eventually dying as a direct consequence. The hallmark of the disease is accumulation of extracellular matrix proteins by pathologically activated fibroblasts. Therapeutic approaches to selectively inhibit the aberrant release of extracellular matrix in SSc are not available to date. Bleomycin-induced dermal fibrosis is the most commonly used mouse model of SSc. It resembles in particular early, inflammatory stages of SSc. Here, we aimed to evaluate the effects of humanized S100A4-antibodies in bleomycin-induced skin fibrosis.

Materials and Methods

Antibody Stock Solution

AX-202, a humanized IgG4 mono-clonal anti-S100A4 antibody, was used for this study. The antibody comprises a heavy chain sequence as defined in SEQ ID NO: 25 and a light chain sequence as defined in SEQ ID NO: 26. The antibody was dissolved in PBS and stored at −20° C.

Study Animals

The experiments were performed on C57Bl/6 mice.

Number of animals: 64

Early mortalities: none

Overall duration of study of each animal: 6 weeks

Treatment Protocol

2 mg/mL antibody stock solution was diluted in sterile PBS and injected i.p. in a volume of 100 μL.

Skin fibrosis was induced by daily subcutaneous injections of bleomycin (2.5 mg/kg, Sigma-Aldrich) in defined and marked areas of the upper back (1 cm²) for up to six weeks. Treatment was commenced after three weeks of pre-challenge with bleomycin, with injections twice weekly intraperitoneally (i.p.) or once every week with intravenous (IV) injection in the tail vein. The outcome was analysed three weeks after the first injection of bleomycin (six weeks after the first bleomycin-injection).

Study Design and Study Groups

N=8 mice in control groups, N=10 mice in treatment groups with the humanized antibody

The following experimental groups were used:

Group 1: Control/NaCl

Group 2: bleomycin 3 weeks and NaCl 3 weeks

Group 3: bleomycin 6 weeks+NaCL for last 3 weeks (every 3rd day IP)

Group 4: bleomycin 6 weeks+AX-202 16 mg/kg for the last 3 weeks (twice a week IP)

Group 5: bleomycin 6 weeks+AX-202 24 mg/kg for the last 3 weeks (weekly IV tail vein injection)

Group 6: bleomycin 6 weeks+AX-202 8 mg/kg for the last 3 weeks (twice a week IP)

Study Conduct

The mice were monitored clinically on a daily basis for behavior, activity, texture of the fur and consistency of the stool. After sacrifice, a gross macroscopic evaluation of the lungs and the skin was performed.

Quantification of Hypodermal Thickening

After sacrifice by cervical dislocation, skin samples of 1 cm2 were obtained from a defined area of on the upper back between the shoulder blades. Lesional skin areas were excised, fixed in 4% formalin for 6 h and embedded in paraffin. Five μm sections were cut and stained with hematoxylin and eosin. The dermal thickness was measured at 100-fold magnification by measuring the distance between the epidermal-dermal junction and the dermal-subcutaneous fat junction at three sites from lesional skin of each mouse.

Detection of Myofibroblasts

Myofibroblasts are characterized by the expression of α-smooth muscle actin (αSMA). Fibroblasts positive for αSMA were detected in paraffin-embedded slides from the upper back by incubation with monoclonal anti-αSMA antibodies (clone 1A4, Sigma-Aldrich, Steinheim, Germany). The expression was visualized with horseradish peroxidase-labelled secondary antibodies and 3,3-diaminobenzidine tetrahydrochloride (DAB) (Sigma-Aldrich). Monoclonal mouse IgG antibodies (Calbiochem, San Diego, Calif., USA) were used for controls. The analysis was performed by a blinded reviewer evaluating the myofibroblasts in four sections per sample.

Hydroxyproline Assay

The amount of collagen protein in skin samples was determined via hydroxyproline assay. After digestion of full skin thickness punch biopsies (0=3 mm) derived from the upper back in 6 M HCl for three hours at 120° C., the pH of the samples was adjusted to 6 with 6 M NaOH. Afterwards, 0.06 M chloramine T was added to each sample and incubated for 20 min at room temperature. Next, 3.15 M perchloric acid and 20% p-dimethylaminobenzaldehyde were added and samples were incubated for an additional 20 min at 60° C. The absorbance was determined at 557 nm with a Spectra MAX 190 microplate spectrophotometer.

Statistics

All data are presented as mean±SEM, and differences between the groups were tested for their statistical significance by one way ANOVA testing using graph pad 8. P-values less than 0.05 were considered significant. P-values are expressed as follows: 0.05>p>0.01 as *; 0.01>p>0.001 as **; p<0.001 as *** as compared to control mice injected with NaCl for 6 weeks. 0.05>p>0.01 as #; 0.01>p>0.001 as ##; p<0.001 as ### as compared to mice injected with bleomycin for 3 weeks followed by injections of NaCl for another 3 weeks.

Results

Dermal Fibrosis Mouse Model

Mice developed prominent dermal fibrosis upon challenge with bleomycin with more pronounced fibrotic changes in mice challenged with bleomycin for 6 weeks as compared to mice challenged with bleomycin for 3 weeks followed by injections of the solvent of bleomycin, NaCl, for another 3 weeks. Mice injected with NaCl for 6 weeks served as controls.

General Tolerability

Treatment with the anti-S100A4 antibody AX-202 was well tolerated without obvious signs of toxicity on clinical examination, on gross necropsy or on histology.

Efficacy Results

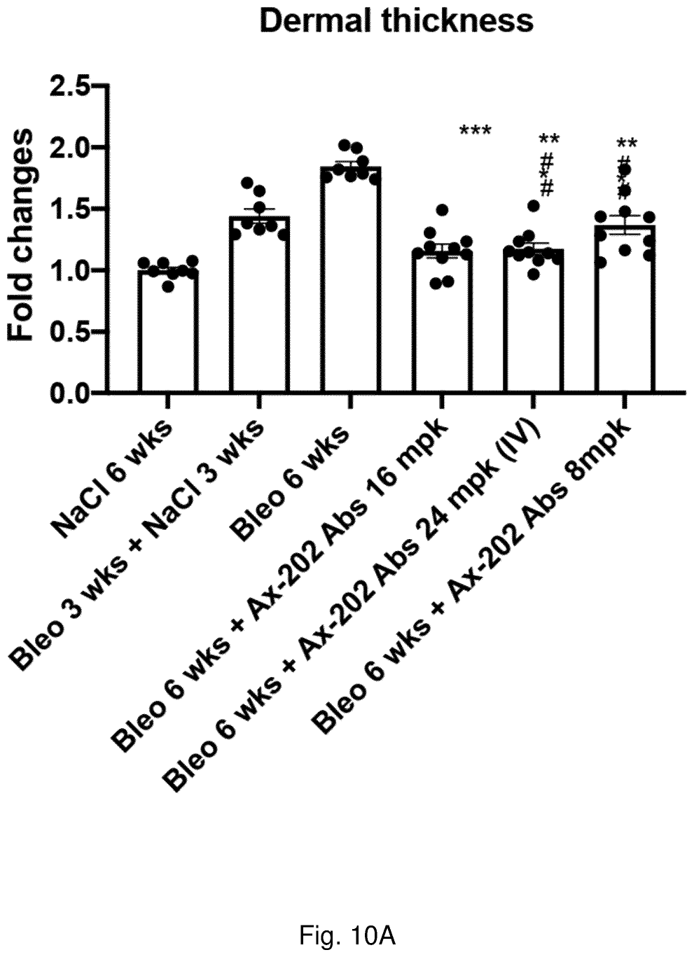

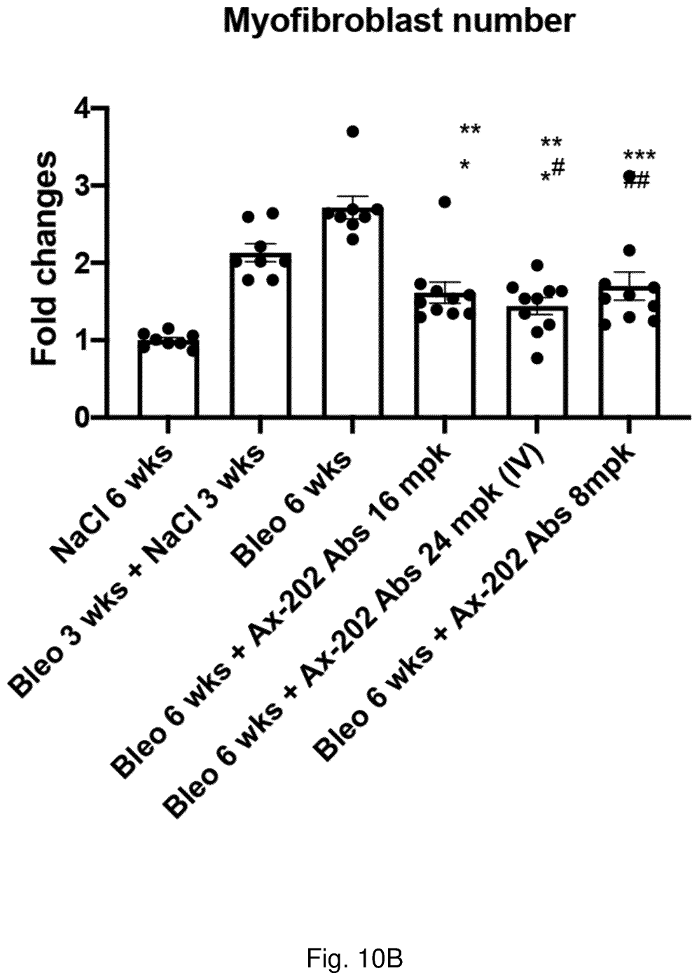

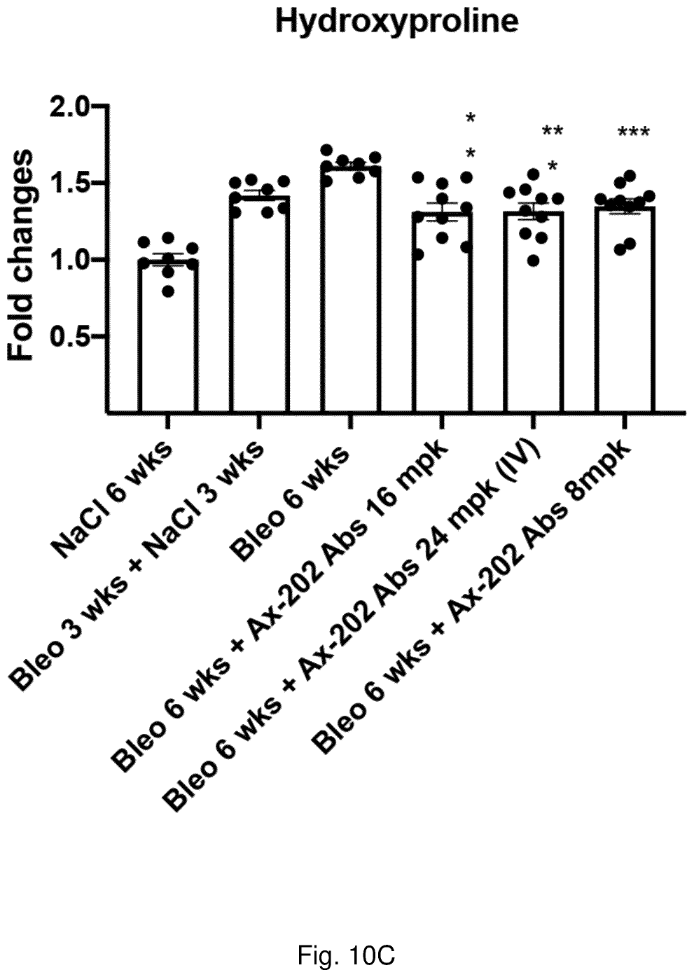

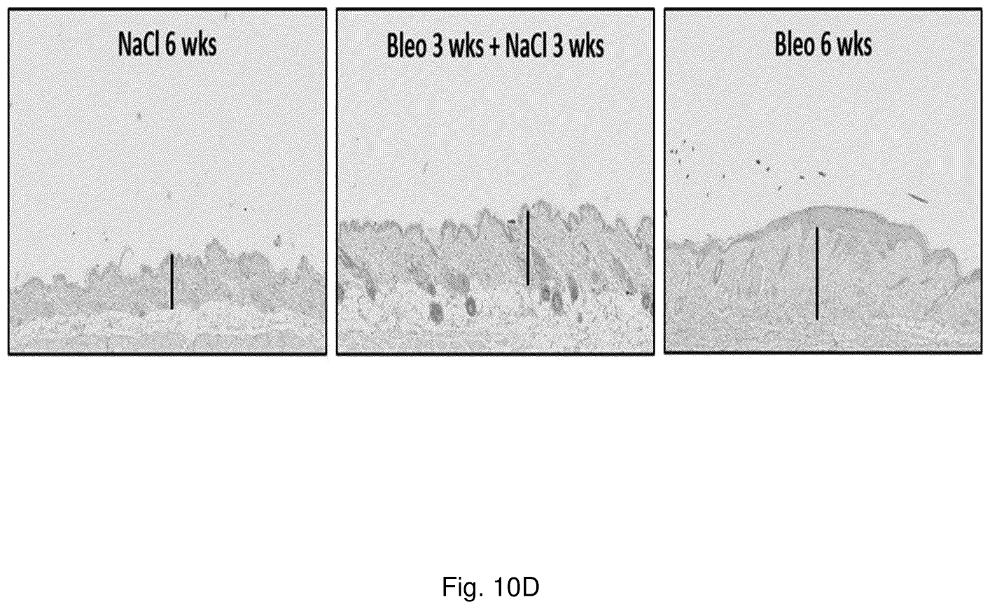

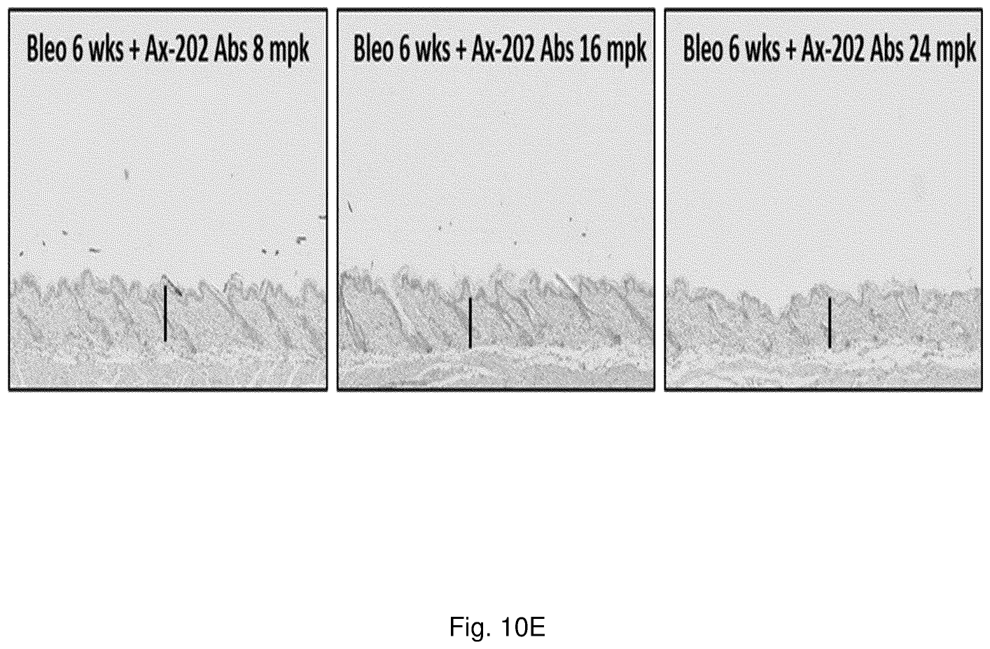

Treatment with AX-202 significantly reduced dermal thickening, myofibroblast counts and the hydroxyproline content as compared to control mice injected with bleomycin for 6 weeks (see FIG. 10). The effects were dose-dependent with most pronounced effects observed in doses of 16 mg/kg IP and 24 mg/kg once weekly IV (see FIGS. 10A-C). However, statistically significant effects of AX-202 were also observed with 8 mg/kg IP every third day. In doses of 16 mg/kg IP and 24 mg/kg IV, AX-202 also induced regression of fibrosis with statistically significant changes of dermal thickness and myofibroblast counts as compared to mice injected with bleomycin only for three weeks.

Conclusion

Treatment with AX-202 strongly ameliorated bleomycin-induced skin fibrosis dermal thickening, myofibroblast counts and hydroxyproline in well-tolerated doses.

### Example 4—Impact on S100A4-Induced Cytokine Release In Vitro

Materials and Methods

Peripheral blood mononuclear cells (PBMCs) were isolated from healthy donors through FicollPaque PLUS (GE Healthcare; 11778538) density centrifugation and monocytes isolated using a monocyte isolation kit (StemCell Technologies; 19359). Monocytes were plated (100,000 cells/well) into 96 well plates and cultured for 6 hours in the presence of:
media,
vehicle 1 (0.074% TBS),
LPS (1.0 ng/ml; Invivogen; tlrl-b5lps),
S100A4 (2.0 μg/ml),
S100A4 (2.0 μg/ml)+vehicle 2 (3.2% PBS),
vehicle 1+mouse IgG1 (Biolegend; 401407),
vehicle 1+human IgG4 (Biolegend; 403701),
vehicle 1+AX-202 or 6B12 (each at 32 μg/ml),
S100A4 (2.0 μg/ml)+mouse IgG1 (at either 8.0, 16 or 32 μg/ml),
S100A4 (2.0 μg/ml)+human IgG4 (at either 8.0, 16 or 32 μg/ml),
S100A4 (2.0 μg/ml)+AX-202 (at either 8.0, 16 or 32 μg/ml), or
S100A4 (2.0 μg/ml)+6B12 (at either 8.0, 16 or 32 μg/ml).

6B12: mouse monoclonal IgG1 anti-S100A4 antibody (VH regions and VL regions as defined in SEQ ID NO: 7 and SEQ ID NO: 9, respectively) AX-202: humanized monoclonal IgG4 anti-S100A4 antibody as described in Example 3

After 6 hours of culture, cell culture supernatant was collected and stored at −20° C. for subsequent cytokine analysis. Levels of cytokines (IL-6, TNF-α and IL-10) in the supernatant were quantified by Luminex assay according to manufacturer's instructions (R&D systems; LXSAHM-03).

Results

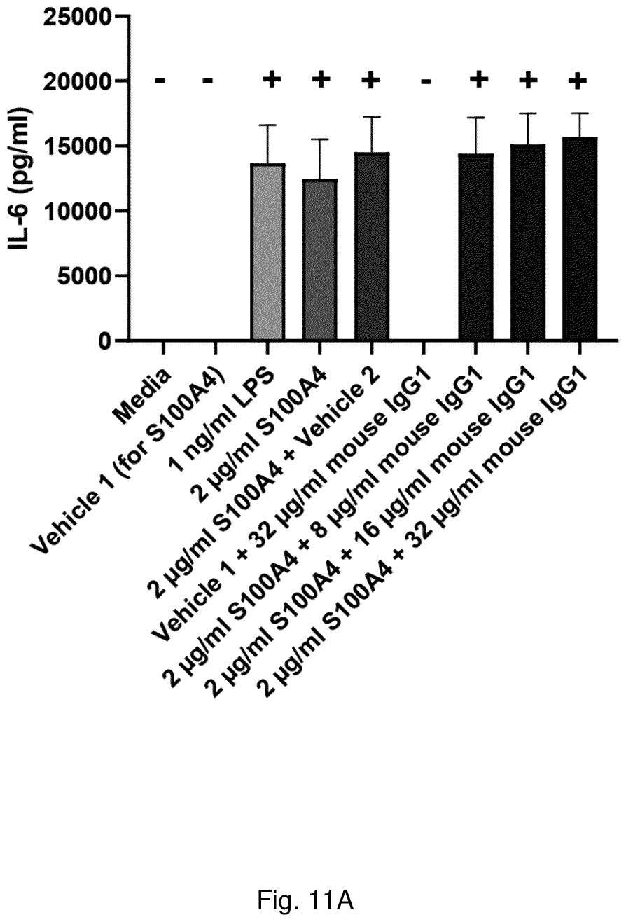

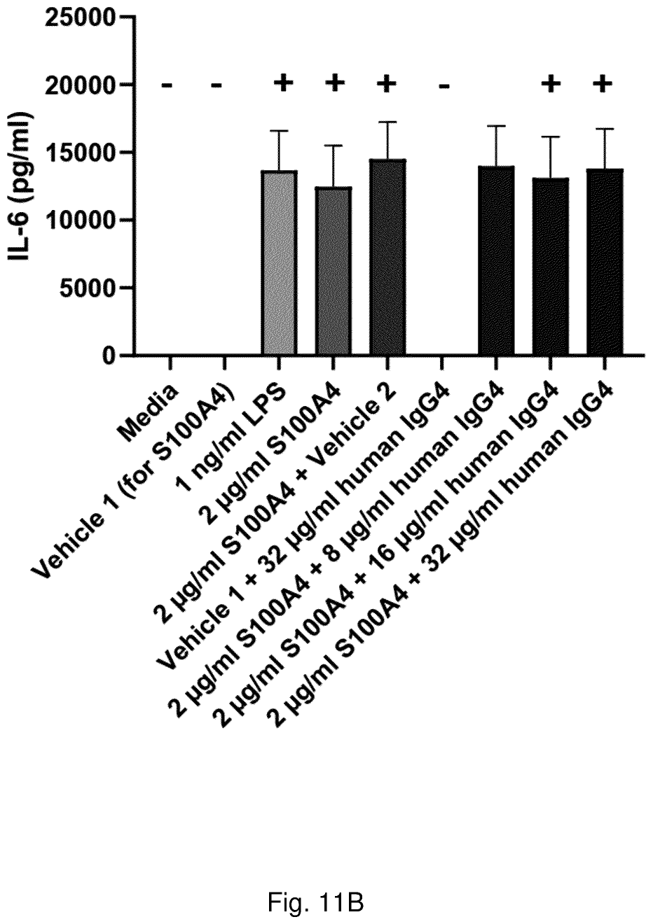

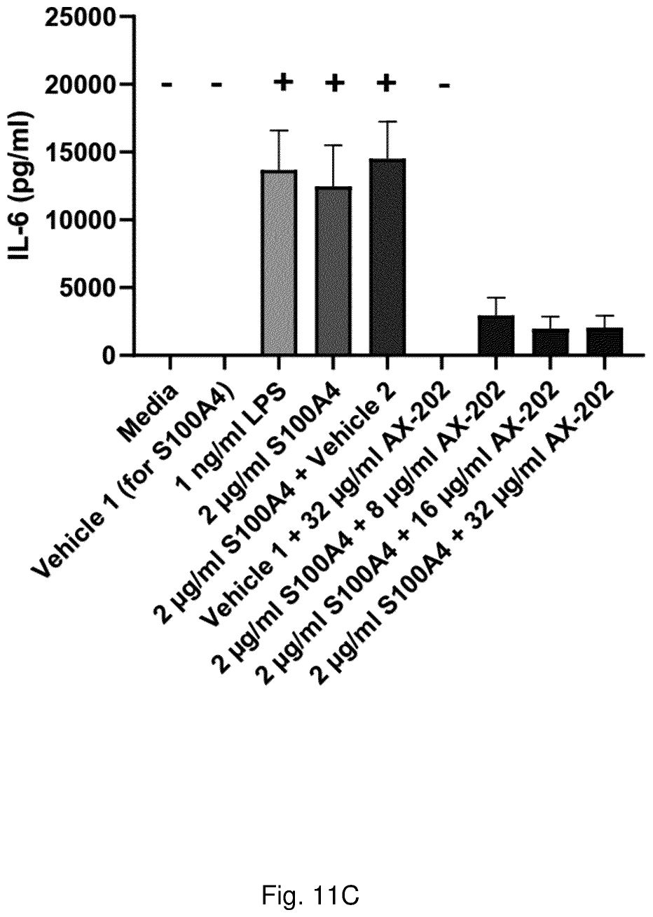

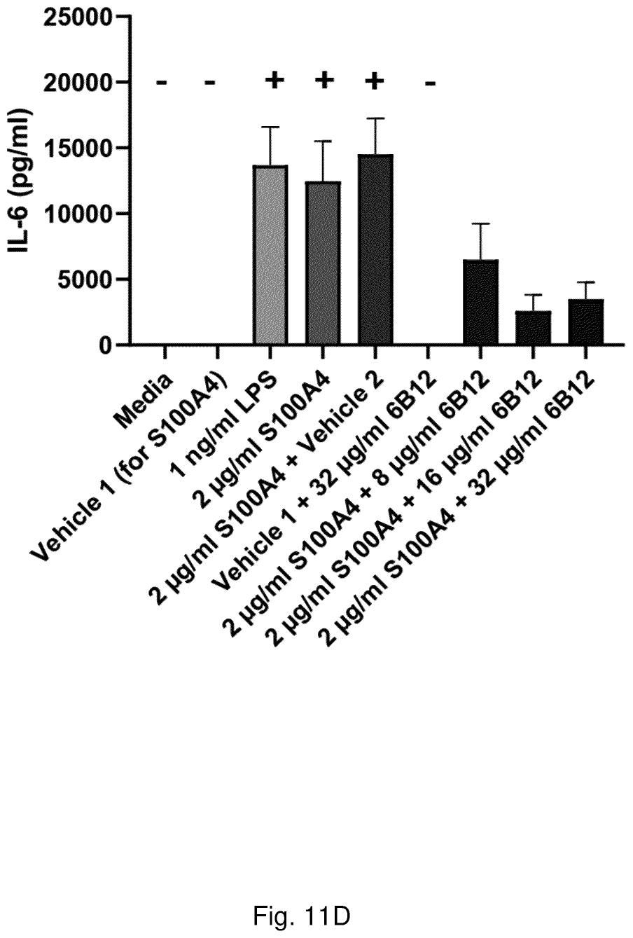

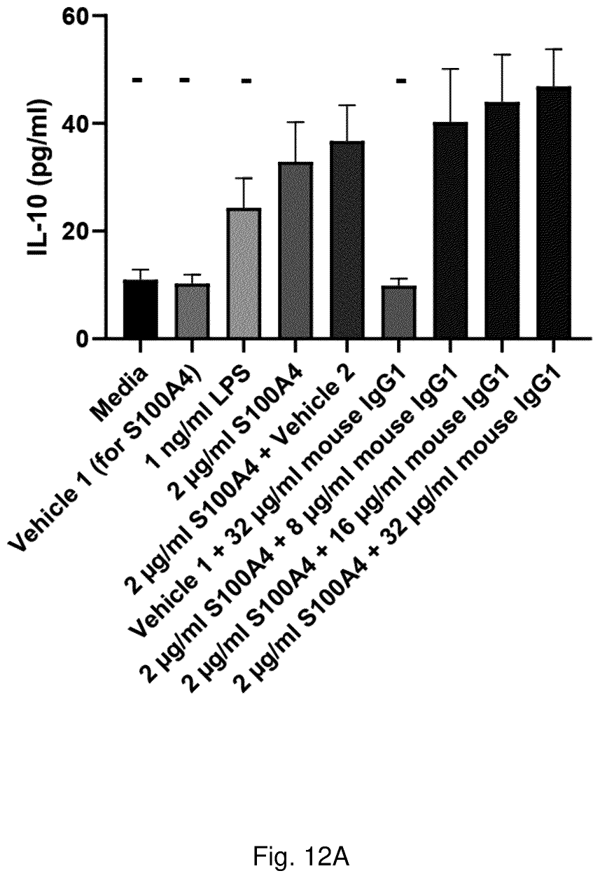

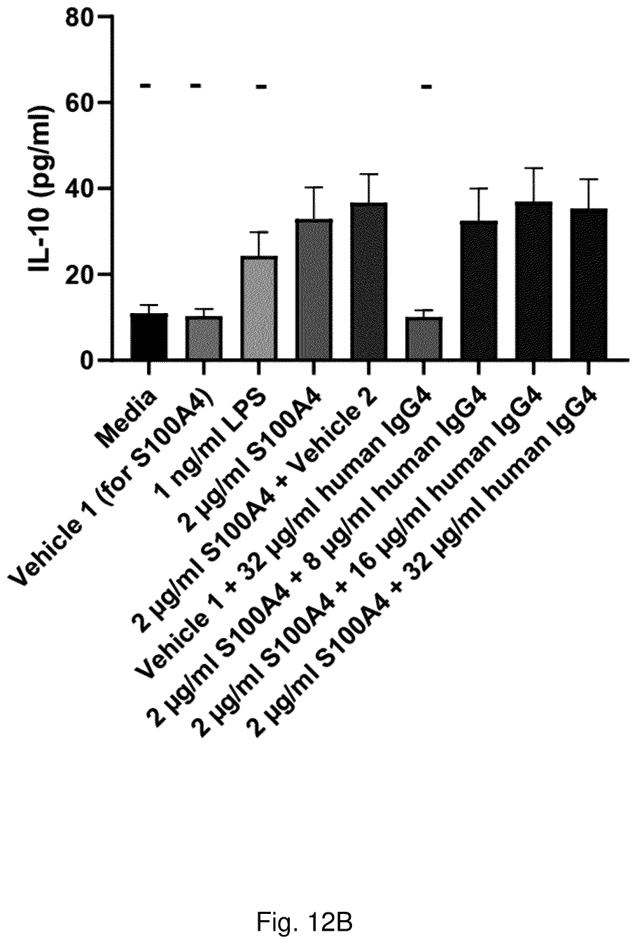

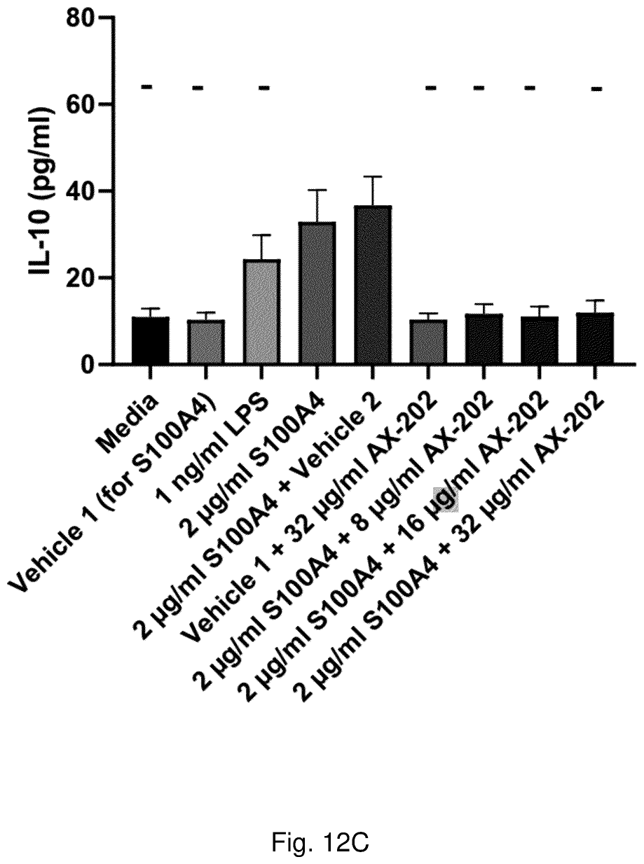

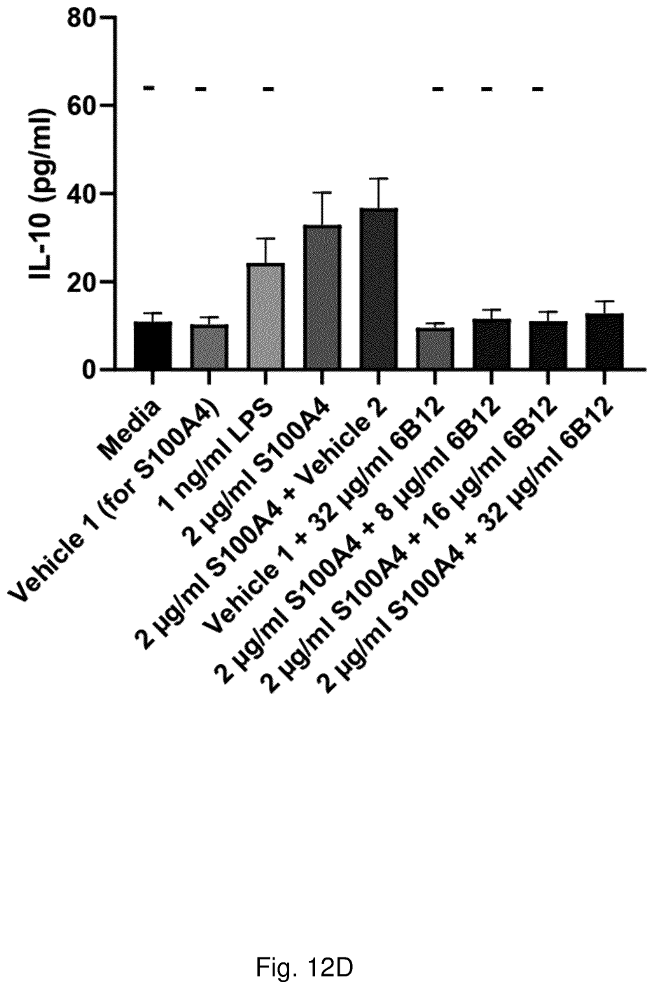

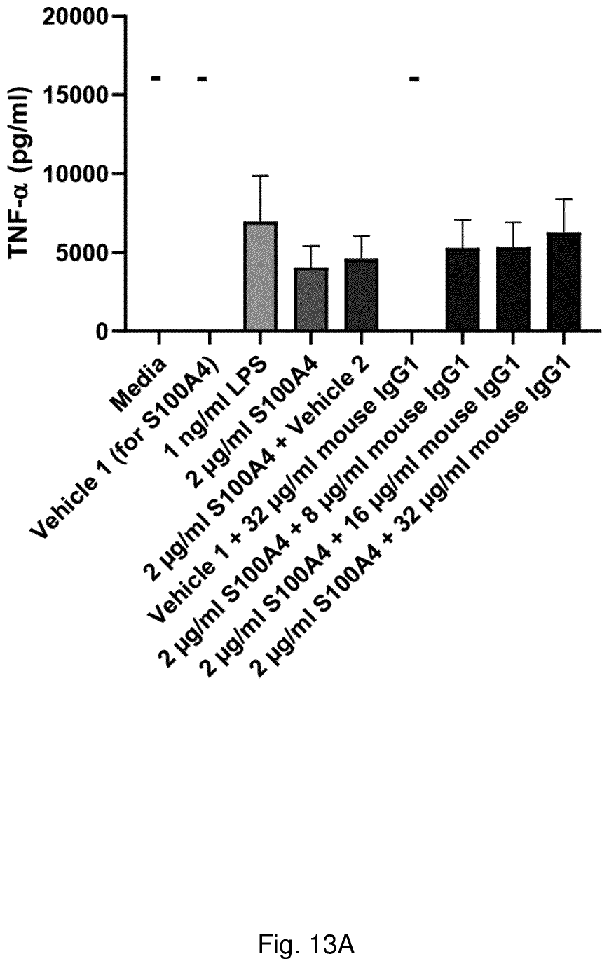

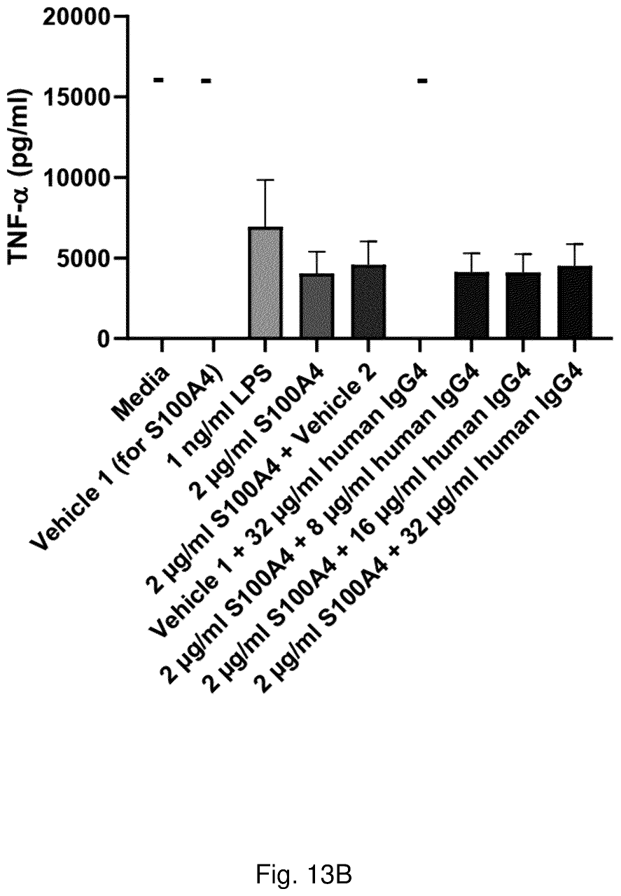

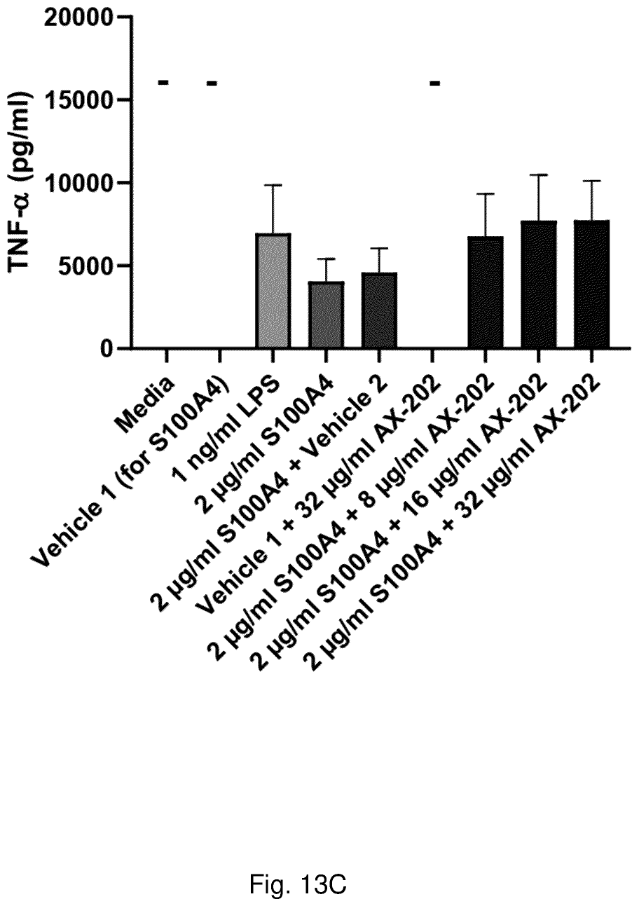

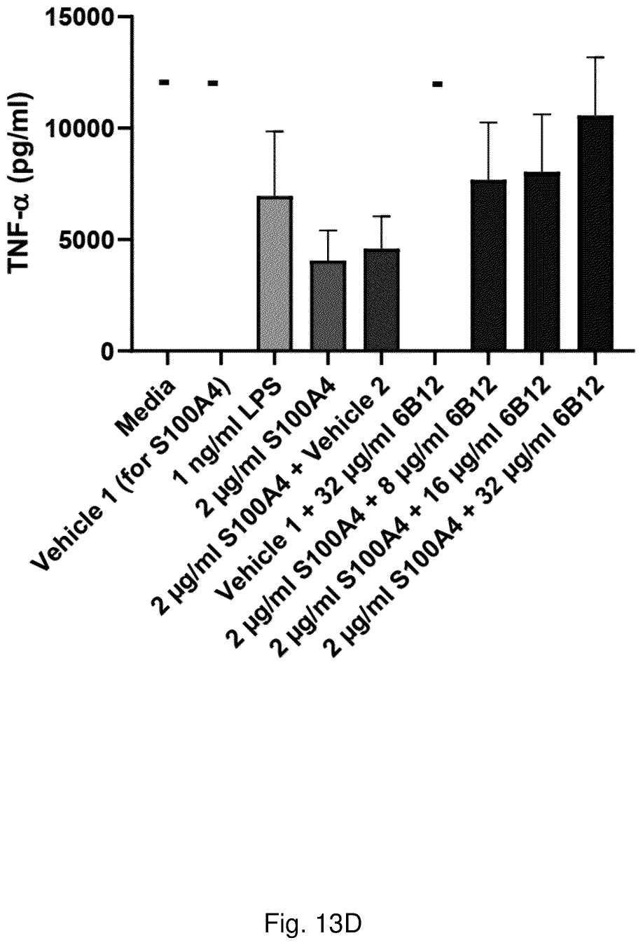

Compared to vehicle control, stimulation of monocytes with either LPS (1.0 ng/ml; positive control) or S100A4 (2.0 μg/ml) evoked an increase in the levels of IL-6, TNFα and IL-10 measured in the cell culture supernatant (see FIGS. 11 and 12). Compared to isotype controls, at all concentrations tested, AX-202 or 6B12 reduced the S100A4-evoked increase in IL-6 and IL-10 (see FIGS. 11C-D and 12C-D). Compared to vehicle controls, S100A4-induced TNFα levels were not increased by mouse IgG1 or human IgG4 isotype controls (see FIGS. 13A-B), however 6B12, and not AX-202, significantly increased S100A4-induced TNFα levels, and the increase induced by 6B12 showed a dose-dependent trend (see FIGS. 13C-D).

Conclusion

As expected, S100A4 evoked an increase in the levels of IL-6, TNFα and IL-10 (see FIGS. 2-4). S100A4-evoked IL-6 and IL-10 release was reduced by both AX-202 and 6B12 (see FIGS. 11C-D and 12C-D).

Importantly, mouse IgG1 control, human IgG4 control, or AX-202 in combination with S100A4 did not result in significant increases in TNFα levels compared to S100A4 alone (see FIGS. 13A-B). In contrast, the levels of S100A4-induced pro-inflammatory cytokine TNFα were increased by treatment with the 6B12 antibody in a dose-dependent fashion (see FIGS. 13C-D).

This indicates that, surprisingly, the humanized anti-S100A4 antibody does not increase S100A4-induced pro-inflammatory TNFα levels compared to the mouse anti-S100A4 antibody 6B12.

Although the foregoing invention has been described in some detail by way of illustration and example for purposes of clarity of understanding, the descriptions and examples should not be construed as limiting the scope of the invention.

The foregoing written specification is considered to be sufficient to enable one skilled in the art to practice the disclosure. It will be appreciated, however, that no matter how detailed the foregoing may appear in text, the disclosure may be practiced in many ways and the disclosure should be construed in accordance with the appended claims and any equivalents thereof.

### REFERENCES

1. Elhai, M. et al. Trends in mortality in patients with systemic sclerosis over 40 years: a systematic review and meta-analysis of cohort studies. (2012). Rheumatology 51, 1017-1026.
2. Varga J, Abraham D. Systemic sclerosis: a prototypic multisystem fibrotic disorder. (2007). J Clin Invest. 117(3):557-67.
3. Brown M, O'Reilly S. Innate immunity and Toll-like receptor signaling in the pathogenesis of scleroderma: advances and opportunities for therapy (2018.) Curr Opin Rheumatol. 30(6):600-605.
4. Eisenbacher J L, Schrezenmeier H, Jahrsdörfer B, Kaltenmeier C, Rojewski M T, Yildiz T, Beyer T, Erle A, Wiegmann D S, Grassl S, Hang R, Körper S, Wiesneth M, Lotze M T, Lotfi R. S100A4 and uric acid promote mesenchymal stromal cell induction of IL-10+/IDO+ lymphocytes. (2014). J Immunol. 192(12):6102-10.
5. Cerezo L A, Remáková M, Tomčik M, Gay S, Neidhart M, Lukanidin E, Pavelka K, Grigorian M, Vencovský J, Šenolt L. The metastasis-associated protein S100A4 promotes the inflammatory response of mononuclear cells via the TLR4 signalling pathway in rheumatoid arthritis. (2014). Rheumatology (Oxford) 53:1520-6.
6. Hansen M T, Forst B, Cremers N, Quagliata L, Ambartsumian N, Grum-Schwensen B, Klingelhöfer J, Abdul-Al A, Herrmann P, Osterland M, Stein U, Nielsen G H, Scherer P E, Lukanidin E, Sleeman J P, Grigorian M. A link between inflammation and metastasis: serum amyloid A1 and A3 induce metastasis, and are targets of metastasis-inducing S100A4. (2015). Oncogene. 34(4):424-35.
7. Neidhart M, Pajak A, Laskari K, Riksen N P., Joosten L A B., Netea G., Lutgens E, Stroes E S G., Ciurea A, Distler O, Grigorian M, Karouzakis E. Oligomeric S100A4 Is Associated With Monocyte Innate Immune Memory and Bypass of Tolerance to Subsequent Stimulation With Lipopolysaccharides (2019). Frontiers in Immunology 10:791 pp.
8. Ambartsumian N, Klingelhöfer J, Grigorian M. The Multifaceted S100A4 Protein in Cancer and Inflammation. (2019). Methods Mol Biol. 1929: 339-365.
9. Klingelhöfer J, Grum-Schwensen B, Beck M K, Knudsen R S, Grigorian M, Lukanidin E, Ambartsumian N. Anti-S100A4 antibody suppresses metastasis formation by blocking stroma cell invasion. (2012). Neoplasia 12: 1260-8.
10. Grum-Schwensen B, Klingelhöfer J, Beck M, Bonefeld C M, Hamerlik P, Guldberg P, Grigorian M, Lukanidin E, Ambartsumian N. S100A4-neutralizing antibody suppresses spontaneous tumor progression, pre-metastatic niche formation and alters T-cell polarization balance. (2015). BMC Cancer 15:44 pp.
11. Bruhn S, Fang Y, Barrends F, Gustafsson M, Zhang H, Konstantinell A, Krönke A, Sönnichsen B, Bresnick A, Dulyaninova N, Wang H, Zhao Y, Klingelhöfer J, Ambartsumian N, Beck M K, Nestor C, Bona E, Xiang Z, Benson M. A generally applicable translational strategy identifies S100A4 as a candidate gene in allergy. (2014). Sci Transl Med. 218:218 pp.
12. Wynn, T. A. Fibrotic disease and the T(H)1/T(H)2 paradigm. (2004.) Nat. Rev. Immunol. 4:583-594.
13. Tan, F. K., et al. Signatures of differentially regulated interferon gene expression and vascu lotrophism in the peripheral blood cells of systemic sclerosis patients. (2006. Rheumatology (Oxford) 45:694-702.
14. Mavalia, C., et al. Type 2 helper T-cell pre-dominance and high CD30 expression in systemic sclerosis. (1997) Am. J. Pathol. 151:1751-1758.
15. Zhang W, Ohno S, Steer B, Klee S, Staab-Weijnitz C A, Wagner D, Lehmann M, Stoeger T, Königshoff M, Adler H. S100a4 Is Secreted by Alternatively Activated Alveolar Macrophages and Promotes Activation of Lung Fibroblasts in Pulmonary Fibrosis. (2018). Front Immunol. 9:1216.
16. Distler J H, Jungel A, Huber L C, Schulze-Horsel U, Zwerina J, Gay R E, et al. Imatinib mesylate reduces production of extracellular matrix and prevents development of experimental dermal fibrosis. (2007) Arthritis Rheum. 56(1):311-322.
17. Gabrielli A, Avvedimento E V, Krieg T. Scleroderma. The New England journal of medicine. 2009 May 7; 360(19):1989-2003.
18. Akhmetshina A, Palumbo K, Dees C, Bergmann C, Venalis P, Zerr P, et al. Activation of canonical Wnt signalling is required for TGF-beta-mediated fibrosis. Nature communications. 2012 Mar. 13; 3:735.
19. Avouac J, Furnrohr B G, Tomcik M, Palumbo K, Zerr P, Horn A, et al. Inactivation of the transcription factor STAT-4 prevents inflammation-driven fibrosis in animal models of systemic sclerosis. Arthritis and rheumatism. 2011 March; 63(3):800-809.
20. Avouac J, Palumbo K, Tomcik M, Zerr P, Dees C, Horn A, et al. Inhibition of activator protein 1 signaling abrogates transforming growth factor beta-mediated activation of fibroblasts and prevents experimental fibrosis. Arthritis and rheumatism. 2012 May; 64(5):1642-1652.
21. Weingartner S, Zerr P, Tomcik M, Palumbo-Zerr K, Distler A, Dees C, et al. Pomalidomide is effective for prevention and treatment of experimental skin fibrosis. Annals of the rheumatic diseases. 2012 November; 71(11):1895-1899.
22. Tomcik M, Palumbo-Zerr K, Zerr P, Avouac J, Dees C, Sumova B, Distler A, Beyer C, Cerezo L A, Becvar R, Distler O, Grigorian M, Schett G, Senolt L, Distler J H. S100A4 amplifies TGF-β-induced fibroblast activation in systemic sclerosis. (2014). Ann Rheum Dis. 74:1748-55.
23. Allanore Y, Simms R, Distler O, Trojanowska M, Pope J, Denton C P, Varga J. Systemic Sclerosis. (2015). Nat Rev Dis Primers. 23; 1:15002.

### SEQUENCES

| | | Organism and | |
|---|---|---|---|
| SEQ | | optionally | |
| ID | | accession | |
| NO: | Description | number | Sequence |
| 1 | CDR-H1 6B12 | Artificial | NDYYWN |
| | monoclonal antibody | sequence | |
| | (amino acid sequence) | | |
| 2 | CDR-H2 6B12 | Artificial | HIGYGGNINYNPSLKN |
| | monoclonal antibody | sequence | |
| | (amino acid sequence) | | |
| 3 | CDR-H3 6B12 | Artificial | ESFYDGYPFDY |
| | monoclonal antibody | sequence | |
| | (amino acid sequence) | | |
| 4 | CDR-L1 6B12 | Artificial | RASQDIRNYLN |
| | monoclonal antibody | sequence | |
| | (amino acid sequence) | | |
| 5 | CDR-L2 6B12 | Artificial | YTSRLHS |
| | monoclonal antibody | sequence | |
| | (amino acid sequence) | | |
| 6 | CDR-L3 6B12 | Artificial | QQGNSLPRT |
| | monoclonal antibody | sequence | |
| | (amino acid sequence) | | |
| 7 | VH region 6B12 | Artificial | MKVLSLLYLLTAIPGILSDV |
| | monoclonal antibody | sequence | QLQESGPGLVKPSQSLSLTC |
| | (amino acid sequence) | | SVTGDSFTNDYYWNWIRQFP |
| | Leader sequence-FR1- | | GSKLEWMGHIGYGGNINYNP |
| | CDR1-FR2-CDR2-FR3- | | SLKNRISITRDTSKNQFFLR |
| | CDR3-FR4 | | LTSVTTEDTATYYCTRESFY |
| | (CDRs underlined, | | DGYPFDYWGQGTLVTVSA |
| | leader sequence in | | |
| | italics, numbering | | |
| | according to the Kabat | | |
| | numbering scheme) | | |
| 8 | VH region 6B12 | Artificial | ATGAAAGTGTTGAGTCTGTT |
| | monoclonal antibody | sequence | GTACCTGTTGACAGCCATTC |
| | (DNA) | | CTGGTATCCTGTCTGATGTA |
| | Leader sequence-FR1- | | CAGCTTCAGGAGTCAGGACC |
| | CDR1-FR2-CDR2-FR3- | | TGGCCTCGTGAAACCTTCTC |
| | CDR3-FR4 | | AGTCTCTGTCTCTCACCTGC |
| | (CDRs underlined, | | TCTGTCACTGGCGACTCCTT |
| | leader sequence in | | CACCAATGATTATTACTGGA |
| | italics, numbering | | ACTGGATCCGGCAGTTTCCA |
| | according to the Kabat | | GGAAGCAAACTGGAATGGAT |
| | numbering scheme) | | GGGCCACATAGGCTACGGCG |
| | | | GTAACATTAACTACAACCCA |
| | | | TCTCTCAAAAATCGAATCTC |
| | | | CATCACTCGTGACACATCTA |
| | | | AGAACCAATTTTTCCTGAGG |
| | | | TTGACTTCTGTGACTACTGA |
| | | | GGACACAGCTACATATTACT |
| | | | GTACAAGAGAGAGTTTCTAT |
| | | | GATGGTTACCCCTTTGATTA |
| | | | CTGGGGCCAAGGGACTCTGG |
| | | | TCACTGTCTCTGCA |
| 9 | VL region 6B12 | Artificial | MMSSAQFLGLLLLCFQGTRC |
| | monoclonal antibody | sequence | DIQMTQTTSSLSASLGDRVT |
| | (amino acid sequence) | | ISCRASQDIRNYLNWYQQRP |
| | Leader sequence-FR1- | | GGTLKLLIYYTSRLHSGVPS |
| | CDR1-FR2-CDR2-FR3- | | RFSGSGSGTDYSLTISNLEQ |
| | CDR3-FR4 | | EDIATYFCQQGNSLPRTFGG |
| | (CDRs underlined, | | GTKLEIK |
| | leader sequence in | | |
| | italics, numbering | | |
| | according to the Kabat | | |
| | numbering scheme) | | |
| 10 | VL region 6B12 | Artificial | ATGATGTCCTCTGCTCAGTT |
| | monoclonal antibody | sequence | CCTTGGTCTCCTGTTGCTCT |
| | (DNA) | | GTTTTCAAGGTACCAGATGT |
| | Leader sequence-FR1- | | GATATCCAGATGACACAGAC |
| | CDR1-FR2-CDR2-FR3- | | TACATCCTCCCTGTCTGCCT |
| | CDR3-FR4 | | CTCTGGGAGACAGAGTCACC |
| | (CDRs underlined, | | ATCAGTTGCAGGGCAAGTCA |
| | leader sequence in | | GGACATTAGGAATTATTTAA |
| | italics, numbering | | ACTGGTATCAGCAGAGACCA |
| | according to the Kabat | | GGTGGAACTCTTAAACTCCT |
| | numbering scheme) | | GATCTACTACACATCAAGAT |
| | | | TACACTCAGGAGTCCCATCA |
| | | | AGGTTCAGTGGCAGTGGGTC |
| | | | TGGAACAGATTATTCTCTCA |
| | | | CCATTAGTAACCTGGAACAA |
| | | | GAAGATATTGCCACTTACTT |
| | | | TTGCCAACAGGGTAATTCGC |
| | | | TTCCTCGGACGTTCGGTGGA |
| | | | GGCACCAAGCTGGAAATCAA |
| | | | A |
| 11 | Human S100A4 | Homo sapiens | MACPLEKALDVMVSTFHKYS |
| | (protein) | (Accession No: | GKEGDKFKLNKSELKELLTR |
| | amino acids 1 to 101 | NP_062427.1) | ELPSFLGKRTDEAAFQKLMS |
| | | | NLDSNRDNEVDFQEYCVFLS |
| | | | CIAMMCNEFFEGFPDKQPRK |
| | | | K |
| 12 | Epitope bound by 6B12 | Homo sapiens | RDNEVDFQEYCVFLSCIAMM |
| | monoclonal antibody | | CNEF |
| | (protein) | | |
| 13 | Epitope bound by 5C3 | Homo sapiens | ELPSFLGKRT |
| | monoclonal antibody | | |
| | (protein) | | |
| 14 | Epitope bound by 5C3 | Homo sapiens | EGFPDKQPRKK |
| | monoclonal antibody | | |
| | (protein) | | |
| 15 | CDR-H1 5C3 | Artificial | ETYMH |
| | monoclonal antibody | sequence | |
| | (amino acid sequence) | | |
| 16 | CDR-H2 5C3 | Artificial | RIDPANGNTKDDPKFQG |
| | monoclonal antibody | sequence | |
| | (amino acid sequence) | | |
| 17 | CDR-H3 5C3 | Artificial | SYAMDY |
| | monoclonal antibody | sequence | |
| | (amino acid sequence) | | |
| 18 | CDR-L1 5C3 monoclonal | Artificial | RSSQSIVHSNGNTYLE |
| | antibody (amino acid | sequence | |
| | sequence) | | |
| 19 | CDR-L2 5C3 monoclonal | Artificial | KVSNRLS |
| | antibody (amino acid | sequence | |
| | sequence) | | |
| 20 | CDR-L3 5C3 monoclonal | Artificial | FQGSHVPFT |
| | antibody (amino acid | sequence | |
| | sequence) | | |
| 21 | VH region 5C3 | Artificial | EAQLQQSGAELVKPGASVKL |
| | monoclonal antibody | sequence | SCTASGFNIQETYMHWVKQR |
| | (amino acid sequence) | | PEQGLEWIGRIDPANGNTKD |
| | FR1-CDR1-FR2-CDR2- | | DPKFQGKASITVDTSSNTAY |
| | FR3-CDR3-FR4 | | LQLSSLTSEDTAVYYCASSY |
| | (CDRs underlined, | | AMDYWGQGTSVTVSS |
| | numbering according to | | |
| | the Kabat numbering | | |
| | scheme) | | |
| 22 | VH region 5C3 | Artificial | GAGGCTCAGCTGCAGCAGTC |
| | monoclonal antibody | sequence | TGGGGCAGAGCTTGTGAAGC |
| | (DNA) | | CAGGGGCCTCTGTCAAGTTG |
| | | | TCCTGCACAGCCTCTGGCTT |
| | | | CAACATTCAAGAGACCTATA |
| | | | TGCACTGGGTGAAGCAGAGG |
| | | | CCTGAACAGGGCCTGGAGTG |
| | | | GATTGGAAGGATTGATCCTG |
| | | | CGAATGGTAATACCAAAGAT |
| | | | GACCCGAAGTTCCAGGGCAA |
| | | | GGCCTCTATAACAGTAGACA |
| | | | CATCCTCCAACACAGCCTAC |
| | | | CTGCAGCTCAGCAGCCTGAC |
| | | | ATCTGAGGACACTGCCGTCT |
| | | | ATTACTGTGCTTCAAGTTAT |
| | | | GCTATGGACTACTGGGGTCA |
| | | | AGGAACCTCAGTCACCGTCT |
| | | | CCTCA |
| 23 | VL region 5C3 | Artificial | DVLMTQTPLSLPVSLGDQAS |
| | monoclonal antibody | sequence | ISCRSSQSIVHSNGNTYLEW |
| | (amino acid sequence) | | YLQKTGQSPELLIYKVSNRL |
| | FR1-CDR1-FR2-CDR2- | | SGVPDRFSGSGSGTDFTLKI |
| | FR3-CDR3-FR4 | | SRVEAEDLGVYYCFQGSHVP |
| | (CDRs underlined, | | FTFGSGTKLEIK |
| | numbering according to | | |
| | the Kabat numbering | | |
| | scheme) | | |
| 24 | VL region 5C3 | Artificial | GATGTTTTGATGACCCAAAC |
| | monoclonal antibody | sequence | TCCACTCTCCCTGCCTGTCA |
| | (DNA) | | GTCTTGGAGATCAAGCCTCC |
| | | | ATCTCTTGCAGATCTAGTCA |
| | | | GAGTATTGTACATAGTAATG |
| | | | GAAACACCTATTTAGAATGG |
| | | | TACCTGCAGAAAACAGGCCA |
| | | | GTCTCCAGAGCTCCTGATCT |
| | | | ACAAAGTTTCCAACCGACTC |
| | | | TCTGGGGTCCCAGACAGGTT |
| | | | CAGTGGCAGTGGATCAGGGA |
| | | | CAGATTTCACACTCAAGATC |
| | | | AGCAGAGTGGAGGCTGAGGA |
| | | | TCTGGGAGTTTATTACTGCT |
| | | | TTCAAGGTTCACATGTTCCA |
| | | | TTCACGTTCGGCTCGGGGAC |
| | | | AAAGTTGGAAATAAAA |
| 25 | AX-202 complete heavy | Artificial | QVQLQESGPGLVKPSQTLSL |
| | chain | sequence | TCTVSGDSFTNDYYWNWIRQ |
| | | | HPGKGLEWIGHIGYGGNINY |
| | | | NPSLKNRLSMSRDTSKNQFS |
| | | | LKLSSVTAADTAVYYCTRES |
| | | | FYDGYPFDYWGQGTLVTVSS |
| | | | ASTKGPSVFPLAPCSRSTSE |
| | | | STAALGCLVKDYFPEPVTVS |
| | | | WNSGALTSGVHTFPAVLQSS |
| | | | GLYSLSSVVTVPSSSLGTKT |
| | | | YTCNVDHKPSNTKVDKRVES |
| | | | KYGPPCPPCPAPEFLGGPSV |
| | | | FLFPPKPKDTLMISRTPEVT |
| | | | CVVVDVSQEDPEVQFNWYVD |
| | | | GVEVHNAKTKPREEQFNSTY |
| | | | RVVSVLTVLHQDWLNGKEYK |
| | | | CKVSNKGLPSSIEKTISKAK |
| | | | GQPREPQVYTLPPSQEEMTK |
| | | | NQVSLTCLVKGFYPSDIAVE |
| | | | WESNGQPENNYKTTPPVLDS |
| | | | DGSFFLYSRLTVDKSRWQEG |
| | | | NVFSCSVMHEALHNHYTQKS |
| | | | LSLSLG |
| 26 | AX-202 complete light | Artificial | DIQMTQSPSSLSASVGDRVT |
| | chain | sequence | ITCRASQDIRNYLNWYQQKP |
| | | | GKALKLLLYYTSRLHSGVPS |
| | | | RFSGSGSGTDYTLTISSLQP |
| | | | EDFATYYCQQGNSLPRTFGG |
| | | | GTKVEIKRTVAAPSVFIFPP |
| | | | SDEQLKSGTASVVCLLNNFY |
| | | | PREAKVQWKVDNALQSGNSQ |
| | | | ESVTEQDSKDSTYSLSSTLT |
| | | | LSKADYEKHKVYACEVTHQG |
| | | | LSSPVTKSFNRGEC |
| 27 | IgG4 CH | Homo sapiens | ASTKGPSVFPLAPCSRSTSE |
| | | | STAALGCLVKDYFPEPVTVS |
| | | | WNSGALTSGVHTFPAVLQSS |
| | | | GLYSLSSVVTVPSSSLGTKT |
| | | | YTCNVDHKPSNTKVDKRVES |
| | | | KYGPPCPPCPAPEFLGGPSV |
| | | | FLFPPKPKDTLMISRTPEVT |
| | | | CVVVDVSQEDPEVQFNWYVD |
| | | | GVEVHNAKTKPREEQFNSTY |
| | | | RVVSVLTVLHQDWLNGKEYK |
| | | | CKVSNKGLPSSIEKTISKAK |
| | | | GQPREPQVYTLPPSQEEMTK |
| | | | NQVSLTCLVKGFYPSDIAVE |
| | | | WESNGQPENNYKTTPPVLDS |
| | | | DGSFFLYSRLTVDKSRWQEG |
| | | | NVFSCSVMHEALHNHYTQKS |
| | | | LSLSLG |
| 28 | κ light chain constant | Homo sapiens | RTVAAPSVFIFPPSDEQLKS |
| | region (CL) | | GTASVVCLLNNFYPREAKVQ |
| | | | WKVDNALQSGNSQESVTEQD |
| | | | SKDSTYSLSSTLTLSKADYE |
| | | | KHKVYACEVTHQGLSSPVTK |
| | | | SFNRGEC |

### ITEMS

1. An anti-human S100A4 antibody for use in the treatment of systemic sclerosis.
2. The antibody for the use according to item 1, wherein the antibody specifically binds to human S100A4 polypeptide of SEQ ID NO: 11, preferably to an epitope contained between amino acids 66 and 89 of SEQ ID NO: 11.
3. The antibody for the use according to any one of the preceding items, wherein the antibody comprises:
   a) a heavy chain variable (VH) region comprising:
      i. a CDR-H1 comprising or consisting of the amino acid sequence of SEQ ID NO: 1;
      ii. a CDR-H2 comprising or consisting of the amino acid sequence of SEQ ID NO: 2; and
      iii. a CDR-H3 comprising or consisting of the amino acid sequence of SEQ ID NO: 3;
and
   b) a light chain variable (VL) region comprising:
      i. a CDR-L1 comprising or consisting of the amino acid sequence of SEQ ID NO: 4;
      ii. a CDR-L2 comprising or consisting of the amino acid sequence of SEQ ID NO: 5; and
      iii. a CDR-L3 comprising or consisting of the amino acid sequence of SEQ ID NO: 6;
or a CDR variant of any one of SEQ ID NO:s 1 to 6, wherein any one amino acid has been altered for another amino acid, with the proviso that no more than 3 amino acids have been so altered, for example wherein 2, or 1 amino acids have been so altered in each CDR.
4. The antibody for the use according to any one of the preceding items, wherein the heavy chain variable (VH) region comprises of SEQ ID NO: 7 or a variant thereof having at least 80% identity, such as at least 85%, such as at least 90%, such as at least 95% identity thereto, and the light chain variable (VL) region comprises of SEQ ID NO: 9 or variant thereof having at least 80% identity, such as at least 85%, such as at least 90%, such as at least 95% identity thereto, preferably wherein the antibody has a VH region defined by SEQ ID NO: 7 and a VL region defined by SEQ ID NO: 9.
5. The antibody for the use according to any one of the preceding items, wherein the antibody comprises:
   a) a heavy chain variable (VH) region comprising:
      i. a CDR-H1 comprising or consisting of the amino acid sequence of SEQ ID NO: 15;
      ii. a CDR-H2 comprising or consisting of the amino acid sequence of SEQ ID NO: 16; and
      iii. a CDR-H3 comprising or consisting of the amino acid sequence of SEQ ID NO: 17;
and
   b) a light chain variable (VL) region comprising:
      i. a CDR-L1 comprising or consisting of the amino acid sequence of SEQ ID NO: 18;
      ii. a CDR-L2 comprising or consisting of the amino acid sequence of SEQ ID NO: 19; and
      iii. a CDR-L3 comprising or consisting of the amino acid sequence of SEQ ID NO: 20;
or a CDR variant of any one of SEQ ID NO:s 15 to 20, wherein any one amino acid has been altered for another amino acid, with the proviso that no more than 3 amino acids have been so altered, for example wherein 2, or 1 amino acids have been so altered in each CDR.
6. The antibody for the use according to any one of the preceding items, wherein the antibody is a bispecific antibody.
7. The antibody for the use according to any one of the preceding items, wherein the antibody is selected from the group consisting of a complete antibody, a Fab fragment, a F(ab′)₂ fragment, a Fab-like fragment, a scFv, a single-domain antibody, a diabody, and a triabody.
8. The antibody for the use according to any one of the preceding items, wherein the antibody molecule is a humanised antibody, a chimeric antibody or a humaneered antibody.
9. The antibody for the use according to any one of the preceding items, wherein the antibody is capable of neutralizing a biological activity of S100A4.
10. The antibody for the use according to any one of the preceding items, wherein the antibody is capable of binding to dimeric, oligomeric and/or multimeric forms of S100A4 protein.
11. The antibody for the use according to any one of the preceding items, wherein the antibody is capable of inhibiting T-cell and/or macrophage recruitment and/or infiltration mediated by S100A4.
12. The antibody for the use according to any one of the preceding items, wherein treatment with the anti-S100A4 antibody reduces fibrosis.
13. The antibody for the use according to item 12, wherein treatment with the anti-S100A4 antibody reduces dermal thickness, dermal collagen or hydroxyproline content, dermal myoblast count and/or T-cell count.
14. The antibody for the use for the use according to any one of the preceding items, wherein the antibody is administered via parenteral administration such as subcutaneously, intramuscularly or intravenously.
15. The antibody for the use according to any one of the preceding items, wherein the antibody is co-administered with a compound selected from the group consisting of an angiotensin-converting enzyme inhibitor, an angiotensin receptor blocker, an azathioprine, a calcium channel blocker, a cyclophosphamide, a hydroxychloroquine, a mycophenolate, a methotrexate, a glucocorticoid, a phosphodiesterase-5 inhibitor, an endothelin receptor antagonist, an alpha blocker, a prostanoid, rituximab, a tyrosine kinase inhibitor such as nintedanib, and tociluzimab.

1. An anti-human S100A4 antibody for use in the treatment of systemic sclerosis, wherein the antibody is capable of neutralizing a biological activity of S100A4.

2. The antibody for the use according to claim 1, wherein the antibody specifically binds to human S100A4 polypeptide of SEQ ID NO: 11, preferably to an epitope contained between amino acids 66 and 89 of SEQ ID NO: 11.

3. The antibody for the use according to any one of the preceding claims, wherein the antibody is capable of binding to an epitope defined by SEQ ID NO: 12.

4. The antibody for the use according to claim 1 or 2, wherein the antibody is capable of binding to an epitope defined by SEQ ID NO: 13.

5. The antibody for the use according to claim 1 or 2, wherein the antibody is capable of binding to an epitope defined by SEQ ID NO: 14.

6. The antibody for the use according to any one of the preceding claims, wherein the antibody comprises:
a) a heavy chain variable (VH) region comprising:
i. a CDR-H1 comprising or consisting of the amino acid sequence of SEQ ID NO: 1;
ii. a CDR-H2 comprising or consisting of the amino acid sequence of SEQ ID NO: 2; and
iii. a CDR-H3 comprising or consisting of the amino acid sequence of SEQ ID NO: 3;
and
b) a light chain variable (VL) region comprising:
i. a CDR-L1 comprising or consisting of the amino acid sequence of SEQ ID NO: 4;
ii. a CDR-L2 comprising or consisting of the amino acid sequence of SEQ ID NO: 5; and
iii. a CDR-L3 comprising or consisting of the amino acid sequence of SEQ ID NO: 6;
or a CDR variant of any one of SEQ ID NO:s 1 to 6, wherein any one amino acid has been altered for another amino acid, with the proviso that no more than 3 amino acids have been so altered, for example wherein 2, or 1 amino acids have been so altered in each CDR.

7. The antibody for the use according to any one of the preceding claims, wherein the heavy chain variable (VH) region comprises of SEQ ID NO: 7 or a variant thereof having at least 80% identity, such as at least 85%, such as at least 90%, such as at least 95% identity thereto, and the light chain variable (VL) region comprises of SEQ ID NO: 9 or variant thereof having at least 80% identity, such as at least 85%, such as at least 90%, such as at least 95% identity thereto, preferably wherein the antibody has a VH region defined by SEQ ID NO: 7 and a VL region defined by SEQ ID NO: 9.

8. The antibody for the use according to any one of the preceding claims, wherein the antibody comprises:
a) a heavy chain variable (VH) region comprising:
i. a CDR-H1 comprising or consisting of the amino acid sequence of SEQ ID NO: 15;
ii. a CDR-H2 comprising or consisting of the amino acid sequence of SEQ ID NO: 16; and
iii. a CDR-H3 comprising or consisting of the amino acid sequence of SEQ ID NO: 17;
and
b) a light chain variable (VL) region comprising:
i. a CDR-L1 comprising or consisting of the amino acid sequence of SEQ ID NO: 18;
ii. a CDR-L2 comprising or consisting of the amino acid sequence of SEQ ID NO: 19; and
iii. a CDR-L3 comprising or consisting of the amino acid sequence of SEQ ID NO: 20;
or a CDR variant of any one of SEQ ID NO:s 15 to 20, wherein any one amino acid has been altered for another amino acid, with the proviso that no more than 3 amino acids have been so altered, for example wherein 2, or 1 amino acids have been so altered in each CDR.

9. The antibody for the use according to any one of the preceding claims, wherein the heavy chain variable (VH) region comprises of SEQ ID NO: 21 or a variant thereof having at least 80% identity, such as at least 85%, such as at least 90%, such as at least 95% identity thereto, and the light chain variable (VL) region comprises of SEQ ID NO: 23 or variant thereof having at least 80% identity, such as at least 85%, such as at least 90%, such as at least 95% identity thereto, preferably wherein the antibody has a VH region defined by SEQ ID NO: 21 and a VL region defined by SEQ ID NO: 23.

10. The antibody for the use according to any one of the preceding claims, wherein the heavy chain comprises or consists of SEQ ID NO: 25 and the light chain comprises or consists of SEQ ID NO: 26.

11. The antibody for the use according to any one of the preceding claims, wherein the antibody is a bispecific antibody.

12. The antibody for the use according to any one of the preceding claims, wherein the antibody is selected from the group consisting of a complete antibody, a Fab fragment, a F(ab′)₂ fragment, a Fab-like fragment, a scFv, a single-domain antibody, a diabody, and a triabody.

13. The antibody for the use according to any one of the preceding claims, wherein the antibody is an immunoglobulin subclass antibody selected from the group consisting of IgG1, IgG2, IgG3 and IgG4.

14. The antibody for the use according to any one of the preceding claims, wherein the antibody is a human IgG4 subclass antibody.

15. The antibody for the use according to any one of the preceding claims, wherein the antibody comprises a human heavy chain constant (CH) region comprising or consisting of the sequence as set forth in SEQ ID NO: 27 or a variant sequence with at least 80%, such as at least 85%, such as at least 90%, such as at least 95% sequence identity thereto.

16. The antibody for the use according to any one of the preceding claims, wherein the antibody comprises an Fc domain with a mutated IgG constant region.

17. The antibody for the use according to any one of the preceding claims, wherein the antibody is PEGylated.

18. The antibody for the use according to any one of the preceding claims, wherein the antibody molecule is a humanised antibody, a chimeric antibody or a humaneered antibody.

19. The antibody for the use according to any one of the preceding claims, wherein the antibody is capable of binding to native conformation S100A4 protein.

20. The antibody for the use according to any one of the preceding claims, wherein the antibody is capable of binding to dimeric, oligomeric and/or multimeric forms of S100A4 protein.

21. The antibody for the use according to any one of the preceding claims, wherein the antibody is capable of inhibiting T-cell and/or macrophage recruitment and/or infiltration mediated by S100A4.

22. The antibody for the use according to any one of the preceding claims, wherein the antibody is capable of inhibiting the biological activity of S100A4 protein in stimulating cell invasion.

23. The antibody for the use according to any one of the preceding claims, wherein treatment with the anti-S100A4 antibody reduces fibrosis.

24. The antibody molecule for the use according to claim 23, wherein fibrosis is assessed by measuring dermal thickness, dermal hydroxyproline content, dermal CD3⁺ cell count and/or dermal myofibroblast counts.

25. The antibody for the use according to any one of the preceding claims, wherein treatment with the anti-S100A4 antibody reduces dermal thickness, dermal collagen or hydroxyproline content, dermal myoblast count and/or T-cell count.

26. The antibody for the use for the use according to any one of the preceding claims, wherein the antibody is administered via parenteral administration such as subcutaneously, intramuscularly or intravenously.

27. The antibody for the use for the use according to any one of the preceding claims, wherein the antibody is administered every week, such as every 2 weeks, such as every 3 weeks, for example every 4 weeks.

28. The antibody for the use for the use according to any one of the preceding claims, wherein the antibody is administered weekly, with a weekly dosage of 25 mg, such as 50 mg, such as 75 mg, such as 100 mg, such as 125 mg, such as 150 mg, such as 175 mg, such as 200 mg, such as 225 mg, such as 250 mg, such as 275 mg, such as 300 mg or more.

29. The antibody for the use according to any one of the preceding claims, wherein the antibody is co-administered with a compound selected from the group consisting of an angiotensin-converting enzyme inhibitor, an angiotensin receptor blocker, an azathioprine, a calcium channel blocker, a cyclophosphamide, a hydroxychloroquine, a mycophenolate, a methotrexate, a glucocorticoid, a phosphodiesterase-5 inhibitor, an endothelin receptor antagonist, an alpha blocker, a prostanoid, rituximab, a tyrosine kinase inhibitor such as nintedanib, and tociluzimab.

30. A pharmaceutical composition for use in the treatment of systemic sclerosis comprising an antibody molecule as defined in any one of claims 1 to 27, and a pharmaceutically acceptable diluent, carrier and/or excipient.

31. An isolated polynucleotide for use in the treatment of systemic sclerosis, which encodes the antibody as defined in any one of claims 1 to 25.

32. A vector comprising the isolated polynucleotide for the use according to claim 31.

33. An isolated host cell comprising the isolated polynucleotide for the use according to claim 31, or the vector for the use according to claim 32.

34. A composition comprising the antibody for the use according to any one of claims 1 to 27, the polynucleotide for the use according to claim 31, the vector for the use according to claim 32 and/or the cell for the use according to claim 33.

35. The composition for the use according to claim 34, wherein the composition is a pharmaceutical composition, further comprising a pharmaceutically-acceptable diluent, carrier and/or excipient.
